(19) United States
(12) Patent Application Publication
(10) Pub. No.: US 2026/0000621 A1
(43) Pub. Date: Jan. 1, 2026

(54) COMPOSITIONS AND METHODS OF USE FOR STABILIZING AND DELIVERY OF ATOMIC LAYER DEPOSITION COATING OF AGENT-CONTAINING LIPID-EMULSION FORMULATIONS

(71) Applicant: THE REGENTS OF THE UNIVERSITY OF COLORADO, A BODY CORPORATE, Denver, CO (US)

(72) Inventors: Theodore W. Randolph, Denver, CO (US); Kendall Preston, Denver, CO (US)

(21) Appl. No.: 19/273,233

(22) Filed: Jul. 18, 2025

Related U.S. Application Data
(63) Continuation of application No. PCT/US2024/012665, filed on Jan. 23, 2024.
(60) Provisional application No. 63/481,182, filed on Jan. 23, 2023.
(60) Provisional application No. 63/482,010, filed on Jan. 27, 2023.

(51) Int. Cl. A61K 9/50; A61K 9/107; A61K 9/14; A61K 39/00; A61K 39/39

(52) U.S. Cl. CPC A61K 9/501; A61K 9/1075; A61K 9/145; A61K 9/5089; A61K 39/39; A61K 2039/55505; A61K 2039/55555; A61K 2039/55566

(57) ABSTRACT

Embodiments of the present disclosure provide novel compositions and methods for making and using thermostable agent-containing lipid-emulsion formulations. In certain embodiments, compositions and methods are disclosed for adapting lipid nanoemulsions of agent-containing formulations having improved stability and/or retaining or enhancing immunogenicity. In other embodiments, agent-containing lipid-nanoemulsion formulations can be spray-dried and further embedded within glassy matrices or microparticles to improve stability and compatibility with other agents by limiting molecular mobility. In other embodiments, these microparticles harboring essentially dried stabilized agent-containing lipid-emulsion formulations can be coated by one or more coating layer to produce stabilized lipid nanoemulsions of agent-containing formulations. In some embodiments, the coating layer is applied by atomic layer deposition (ALD) and the coating layers include a metalloorganic material.

### CROSS-REFERENCED APPLICATIONS

This Continuation Application claims priority to International Application PCT/US2024/012665 filed Jan. 23, 2024, which claims priority to U.S. Provisional Application No. 63/482,010 filed Jan. 27, 2023, and U.S. Provisional Application No. 63/481,182 filed Jan. 23, 2023. These applications are incorporated herein by reference in their entireties for all purposes.

### FIELD

Embodiments of the present disclosure provide novel compositions and methods for making and using thermostable lipid emulsion agent-containing formulations. In certain embodiments, compositions and methods are disclosed for adapting lipid nanoemulsions of agent-containing formulations having improved stability and/or retaining or enhancing immunogenicity. In other embodiments, lipid nanoemulsions of agent-containing formulations can be spray-dried and further embedded within glassy matrices or microparticles to improve stability and compatibility with other agents by limiting molecular mobility. In other embodiments, these microparticles harboring essentially dried stabilized lipid nanoemulsions of agent-containing formulations can be coated by one or more coating layer to produce stabilized lipid nanoemulsions of agent-containing formulations.

### BACKGROUND

Therapeutic impacts of vaccines or other antigens can be compromised by many challenges. One challenge is accurate and reliable delivery of a vaccine to a subject. For example, multiple administrations are often required to provide successful immunity to a particular pathogen or against another health disorder such as cancer, which can decrease the likelihood that a subject will or can take the necessary steps to obtain a required second or more administrations. In addition, vaccines and vaccine formulations often exhibit increased instability during storage, transport and handling, and the vaccine supply chain can require expensive and logistically complex refrigeration (e.g., cold chain requirements to reduce adverse effects of elevated temperatures). Further, certain vaccines against a particular pathogen must be separately administered at the same time or at different times to a subject to avoid being ineffective when combined with a different incompatible vaccine against another pathogen creating additional expenses and the need for different administration to avoid adverse effects. Additionally, vaccines and vaccine formulations are typically associated with high manufacturing, storage, and delivery costs, which can limit their availability to those in need.

### SUMMARY

Embodiments of the present disclosure provide novel compositions and methods for making and using thermostable lipid emulsion therapeutic agent-containing formulations. In certain embodiments, compositions and methods are disclosed for adapting lipid nanoemulsions of therapeutic agent-containing formulations for improved stability and/or retaining or enhancing immunogenicity or efficacy of the therapeutic agent. In other embodiments, lipid nanoemulsions of therapeutic agent-containing formulations can be spray-dried for embedding the therapeutic agent in a glassy matrix or microparticle to improve stability and compatibility with other therapeutic agents. In accordance with these embodiments, molecular mobility can be limited thereby reducing adverse reactions, reducing degradation, and/or reducing therapeutic agent interactions. In other embodiments, these microparticles harboring essentially dried stabilized lipid nanoemulsions of therapeutic agent-containing formulations can be coated by one or more coating layer to produce stabilized lipid nanoemulsions of agent-containing formulations for delayed or timed delivery in a subject. In some embodiments, the coating layer can be applied by atomic layer deposition (ALD) and the coating layers can include a metalloorganic material such as a metal oxide or metal alkoxide.

In certain embodiments and further to paragraph above, spray-dried nanoemulsified lipid therapeutic agent-containing formulations disclosed herein can form essentially dried microparticles in the form of powders where these microparticles including the essentially dried nanoemulsified lipid therapeutic agent-containing formulations can be introduced to a fluidized bed atomic layer deposition (ALD) reactor disclosed herein to apply one or more coating layers. In accordance with these embodiments, each coating layer of the one or more coating layers can include one or more of a metallo-organic material, metal oxides, metal alkoxides, and/or aluminum-based coating layer. In certain embodiments, the coating layer can include, but is not limited to, a composition including, aluminum oxide (Al₂O₃), an aluminum alkoxide, silicon dioxide (SiO₂), titanium dioxide (TiO₂), Zinc dioxide (ZnO₂) and silicon nitride (Si₃N₄). In certain embodiments, the coating layer can include, but is not limited to mixtures of two or more of aluminum oxide (Al₂O₃), an aluminum alkoxide, silicon dioxide (SiO₂), titanium dioxide (TiO₂), Zinc dioxide (ZnO₂) and silicon nitride (Si₃N₄) compositions in the same, alternating, or other pattern of layering such as 1:2, 1:3, 1:4, 1:5 or other pattern (e.g., alternating layers of aluminum agents with silicon-or titanium-containing compositions, for example). In accordance with these embodiments, combinations of spray-drying of the nanoemulsified lipid therapeutic agent-containing formulations and then ALD-coating the essentially dry nanoemulsified lipid therapeutic agent-containing formulations to increase thermostability, reduce incompatibility of therapeutic agents and provide delayed or timed-release of the nanoemulsified lipid therapeutic agent-containing formulations. For example, these coated essentially dry nanoemulsified lipid therapeutic agent-containing formulations have improved stability at room temperature or higher temperature storage (e.g., 40° C. to about 70° C.).

In certain embodiments and further to paragraphs [0004]-[0005] above, ALD coated microparticles harboring or containing lipid-emulsion therapeutic agent-containing formulations (e.g., lipidic adjuvant suspensions and/or lipid nanoparticles) can lead to increased stability and/or compatibility of an antigen or other agent of the coated and stabilized lipid-containing therapeutic agent-containing nanoemulsions to provide for timed-release delivery of the therapeutic antigen or therapeutic agent contained within the coated lipid-emulsion therapeutic agent-containing microparticles, increased compatibility between therapeutic agents if more than one therapeutic agent or antigen is to be contained within the coated microparticles, and reduced concentrations of therapeutic agents and/or antigens needed to treat, reduce onset of, ameliorate or prevent onset of a health condition.

In some embodiments and further to paragraphs [0004]-[0006] above, primary and at least one boost dose of coated lipid-emulsion therapeutic agent-containing or lipid-suspension-therapeutic agent-containing microparticle formulations can be administered to a subject in a single administration of these coated microparticles. In other embodiments, coated lipid-emulsion, or lipid-suspension therapeutic agent-containing microparticles disclosed herein can include antigens (e.g., immunogenic antigens) or agents against two or more pathogens or other essentially dried lipid-emulsion or lipid-suspension therapeutic agent-containing formulations in the same or in separate microparticles.

In certain embodiments and further to paragraphs [0004]-[0007] above, compositions and methods disclosed herein describe critical formulations, parameters, and methods for spray-drying of lipid emulsified adjuvant-, antigen-, and/or therapeutic agent-containing formulations using a squalene-based nanoemulsion system for developing thermostable lipid emulsified adjuvant-, antigen-, and/or therapeutic agent-containing formulations. In certain embodiments, much of the bulk liquids or water of these formulations can be removed to attain a formulation with residual moisture of about 5.0% w/v or less, about 2.5% w/v or less, about 1.0% w/v or less, about 0.5% w/v or less to reduce or restrict molecular mobility of a nanoemulsion or nanosuspension lipid adjuvant-, antigen-, and/or therapeutic agent-containing composition. In accordance with these embodiments, an emulsified lipid adjuvant-, antigen-, and/or therapeutic agent-containing composition or formulation having reduced water content has dramatically reduced or restricted molecular mobility leading to reduced collision frequencies between emulsified nanodroplets or suspended nanoparticles and/or lower rates of nanodroplet coalescence or nanoparticle agglomeration or the like. In accordance with these embodiments, reducing collision frequency between emulsified nanodroplets or suspended nanoparticles by reducing water or moisture content of a lipid emulsion adjuvant-, antigen-, and/or therapeutic agent-containing formulation can lead to stability at temperatures from about 35° C., up to 60° C. or up to 70° C. or higher. In other embodiments, reduced water or moisture content of these compositions and stabilization at increased temperatures can permit coating of these reduced moisture-containing agent-or antigen-containing lipid emulsion compositions.

In some embodiments and further to paragraphs [0004]-[0008] above, to create a reduced-moisture or reduced water-containing lipid-emulsion or lipid-suspension adjuvant-, antigen-, and/or therapeutic agent-containing formulations, a core of these compositions in essentially dried form can be formed by spray-drying in formulations containing one or more salt, one or more glass-forming polysaccharides (e.g., trehalose, sucrose, glycine and mannitol, other disaccharide, or similar) and a high molecular weight glass-forming agent or smoothing agent (e.g., hydroxyethyl starch, dextran, carboxymethyl cellulose, or the like). In accordance with these embodiments, in liquid formulations prior to spray drying or dehydrating, salt concentrations can be about 0.1 mM to about 250.0 mM (or about 10 mM to about 200 mM or about 80 mM or less); polysaccharide concentrations can be about 2.5% to about 30.0% (or about 7.50% to about 25.0%, or about 7.50% to about 20.0%) and a high molecular weight glass-forming agent such as an additional glass-forming agent concentration, can be about 0.1% to about 10.0%, or about 1.0% to about 5.0%. In certain embodiments, a formulation prior to spray-drying disclosed herein contains about 0.1% to about 10.0%, or about 1.0% to about 5.0% hydroxyethyl starch, about 2.5% to about 30.0% (or about 7.50% to about 25.0% or about 7.50% to about 20.0%) sucrose and/or about 2.5% to about 30.0% (or about 7.50% to about 25.0% or about 7.50% to about 20.0%) trehalose. In some embodiments, surprisingly, it was discovered that even though adjuvant-, antigen-, and/or therapeutic agent-containing lipid emulsions could be, or were unstable in liquid solutions, when formulated at the same high salt concentrations found during spray drying (e.g., about 10 mM to about 80 mM, or about 50 mM to about 1000 mM or about 150 mM to about 640 mM), salt-induced instabilities of these emulsions were reduced or eliminated by co-formulating the lipid emulsions with at least one glass-forming polysaccharide (e.g., hydroxyl starch, sucrose, trehalose or other comparable polysaccharide). In yet other embodiments, instabilities could be further reduced or eliminated if the one or more polysaccharide agents were combined with the lipid-emulsion adjuvant-, antigen-, and/or therapeutic agent-containing formulation prior to spray-drying in the presence of solutions containing high concentrations of polysaccharides (e.g., 0.5 wt % to about 25.0 wt % or about 2.0 wt % to about 15.0 wt %), where the combination of elevated polysaccharide concentrations and rapid (e.g. about a few seconds to sub-second timing) spray-drying times combine to reduce both droplet-droplet collision frequencies and the time available for these collisions to result in undesirable coalescence or agglomeration of the adjuvant-, antigen-, and/or therapeutic agent-containing lipid emulsions or suspensions, leading to stabilized spray-dried formulations. In certain embodiments, quick spray-drying while introducing elevated (e.g., 0.5 wt % to about 25.0 wt % or about 2.0 wt % to about 15.0 wt %) polysaccharide concentrations to these formulations creates a microparticle core (e.g., a powder) coatable by ALD or similar system for coating these stabilized microparticles (e.g., applying a metallo-organic, metal oxide agent, metal alkoxide or the like).

In some embodiments and further to paragraphs [0004]-[0009] above, stabilized lipid-emulsion adjuvant-, antigen-, and/or therapeutic agent-containing powders or glassy matrices or microparticles to be coated with one or more coating layers where each layer of the one or more coating layers can include one or more of a metallo-organic material, metal oxides, metal alkoxides, and/or aluminum-based coating layer. As disclosed herein, the term particle or particles, microparticles, nanoparticles can be referred to simply as particles or the like and can be used interchangeably without interpretation as to size, sizes of particles are contemplated herein to be of a size that is capable of being administered by a syringe or being part of atomizable, inhalable, or topically appliable forms as appropriate given the health condition to be treated or prevented. In certain embodiments, coating materials for layering onto stabilized particles disclosed herein can include, but are not limited to, aluminum oxide (Al₂O₃), an aluminum alkoxide, silicon dioxide (SiO₂), titanium dioxide (TiO₂), Zinc oxide (ZnO₂), and silicon nitride (Si₃N₄). In accordance with these embodiments, combinations of spray-drying of the nanoemulsified or nano-suspended lipid formulations and ALD-coating of the essentially dry nanoemulsified or nano-suspended lipid microparticles increases the thermostability of the nanoemulsified or nano-suspended adjuvant-, antigen-, and/or therapeutic agent-containing lipid microparticles. For example, these coated essentially dry nanoemulsified or nano-suspended microparticles have improved stability at room temperature storage or higher temperature storage. It was surprisingly observed that spray-dried lipid-emulsion adjuvant-, antigen-, and/or therapeutic agent-containing formulations form microparticles that can be introduced to a fluidized bed ALD reactor and stabilized microparticle surfaces coated without altering the size distribution of the embedded lipid nanoemulsion or nano-suspension adjuvant-, antigen-, and/or therapeutic agent-containing microparticles contained with the powder or essentially-dried formulation. In certain embodiments, coated lipid-emulsion adjuvant-, antigen-, and/or therapeutic agent-containing powders or glassy matrices can be stored, transported, and reconstituted for use to treat, reduce onset, or prevent a medical condition. In accordance with these embodiments, reconstituted coated microparticles maintain size distributions that are syringable, atomizable, and/or inhalable and capable of being introduced to a subject by any delivery method known in the art.

In certain embodiments and further to paragraphs [0004]-[0010] above, the antigen-and/or therapeutic agent is at least one immunogenic agent that forms part of a central or innermost coated microparticle including at least one immunogenic agent and at least one glass-forming agent, and one or more outer coating layers covering or encasing the central glassy microparticle. In accordance with these embodiments, 1, 2, 3, 4, 5, up to 10, up to 20, up to 30, up to 40, up to 100, up to 150, up to 200, up to 250 or more coating layers can encase stabilized lipid-emulsion antigen-, and/or therapeutic agent-containing powders or glassy matrices or microparticles where the coating layers are readily dissolvable in a subject, once administered to the subject, to expose the one or more therapeutic agent or antigen to the subject by immediate exposure, delayed release and/or timed-release. In other embodiments, the at least one immunogenic agent or immunogenic antigen can be stabilized in powder form as disclosed herein and encased in another layer of coating agent forming a prime dose of the at least one immunogenic agent or immunogenic antigen to be exposed prior to dissolution of the microparticles to expose the inner core. In yet other embodiments, the at least one immunogenic agent or immunogenic antigen can include a mixture of immunogenic agent or immunogenic antigen, or separate immunogenic agent or immunogenic antigen in the inner core from the same or different immunogenic agents mixed with coating layers forming at least one outer layer of coated microparticles for differential exposure of the at least one immunogenic agent or immunogenic antigen.

Certain embodiments and further to paragraphs [0004]-[0011] above, the present disclosure provides methods for making stabilized lipid-emulsion adjuvant-, antigen-, and/or therapeutic agent-containing coated microparticles, stabilized powders or glassy matrices, the method including combining at least one lipid emulsion of at least one adjuvant, antigen, and/or therapeutic agent with at least one glass-forming agent to form a primary liquid composition, rapidly spray-drying the composition to form lipid emulsion adjuvant-, antigen-, and/or therapeutic agent-containing glassy microparticles, and coating the essentially-dried lipid emulsion adjuvant-, antigen-, and/or therapeutic agent-containing glassy microparticles with one or more outer coating layers. In some embodiments, the primary liquid composition can further include a second glass forming agent or high molecular weight glass forming agent. In accordance with these embodiments, the second glass-forming agent can include hydroxyethyl starch or similar agent. Further, in accordance with these embodiments, hydroxyethyl starch precipitates as a film on the surface of droplets of the composition as they dry during spray drying, reducing agglomeration or coalescence of microparticles thereby maintaining integrity of the particles as they dry to a more stabilized state in preparation for coating by ALD or other metallo-organic layering application system.

In accordance with these embodiments and further to paragraphs [0004]-[0012] above, the at least one adjuvant, antigen, and/or therapeutic agent can include one or more adjuvant, antigen, and/or therapeutic agent, including, but not limited to, a viral antigen, a bacterial antigen, a toxin, a prion, yeast, a fragment or subunit thereof, a chemical agent, a small molecule, an anti-cancer agent, an anti-inflammatory agent, an anti-autoimmune agent, a peptide, polynucleotide, or protein thereof or a combination thereof. In some embodiments, the at least one agent or antigen can include one or more agent(s) or antigen(s) including, but is not limited to, a recombinant peptide, a recombinant protein, a peptide derived from a target protein or pathogen, a polysaccharide derived from a target pathogen, a synthetic peptide or protein, a virus-like particle, a live virus, a live, attenuated virus, an inactivated virus, an antigen attached to, associated with, or expressed on the surface of a virus or a bacteriophage or a combination thereof. In yet other embodiments, the at least one agent or antigen can include one or more agent(s) or antigen(s) represented by a polynucleotide, encoded by a polynucleotide or the like. In accordance with these embodiments, a polynucleotide can include, but is not limited to, DNA, RNA, mRNA, siRNA, or a chimeric molecule thereof. In certain embodiments, a chimera can include a combination of at least one polynucleotide segment and at least one polypeptide segment or a mixture of polynucleotide segments and/or polypeptide segments. In some embodiments, the polynucleotide can be mRNA encoding a full length or peptide fragment of a targeted agent (e.g., virus or bacteria).

In certain embodiments and further to paragraphs [0004]-[0013] above, the at least one agent can include, but is not limited to, at least one pathogen or antigen derived therefrom for example, antigens derived from human papilloma virus (e.g., HPV16, HPV18 or other serotype or type), or other mammalian papilloma virus, ricin toxin, Bacillus anthracis, Clostridium botulinum, Ebola virus, influenza virus, Corona virus (SARS-COV-2) or other variants or mutants thereof, SARS or other variants, poliovirus, norovirus, rotavirus, hepatitis C, varicella, herpes simplex, cytomegalovirus, Japanese encephalitis, dengue virus, West Nile virus, Zika virus or other flaviviruses, Chikungunya, Equine Encephalitis virus (EEV) or other alphavirus, pneumonia virus spp., Yersinia, Pneumococcus, Salmonella, Clostridium difficile, or a combination thereof. In certain embodiments, the at least one antigen or agent can be a multimeric complex. In other embodiments, the at least one antigen or agent can include a pathogen or antigen derived therefrom capable of infecting a human, companion animal, livestock, wild animal, zoo animal, bird, fish, or reptilc.

In some embodiments and further to paragraphs [0004]-[0014] above, the at least one glass-forming agent or polysaccharide or disaccharide disclosed herein can include at least one of trehalose, sucrose, ficoll, dextran, sucrose, maltotriose, lactose, mannitol and sucrose, hydroxyethyl starch, glycine, cyclodextrin, povidone, or the like. In certain embodiments, the at least one glass-forming agent or polysaccharide disclosed herein can include trehalose or sucrose. In certain embodiments, the at least one glass-forming agent or polysaccharide disclosed herein can include hydroxyethyl starch alone or in combination with a second polysaccharide. In accordance with these embodiments, the at least one glass-forming agent or polysaccharide disclosed herein can be present in the primary liquid immunogenic composition in a weight-to-volume (w/v) concentration of from about 0.1% to about 40.0%, from about 1.0% to about 30.0%, from about 5.0% to about 20.0%, or from about 8.0% to about 15.0%. In certain embodiment, the at least one glass-forming agent or polysaccharide disclosed herein can be combined with the lipid-emulsion agent(s) or antigen and further spray-dried and further used in a formulation or in preparation for application of one or more coatings to the essentially dried microparticles formed by this process for later formulation and uses disclosed herein.

In other embodiments and further to paragraphs [0004]-[0015] above, the glass-forming agent or polysaccharide disclosed herein can include at least one additional polysaccharide. In accordance with these embodiments, the at least one additional polysaccharide can be a hydroxyethyl starch or other pharmacologically acceptable plasma expander such as human serum albumin (HSA), other serum albumins, dextran, hetastarch, plasma protein factor and the like, or a combination thereof. In accordance with these embodiments, the at least one additional polysaccharide can be present in a primary formulation prior to spray-drying at a weight-to-volume (w/v) concentration from about 0.1% to about 40.0%, from about 1.0% to about 30.0%, from about 5.0% to about 20.0%, or from about 8.0% to about 15.0%. In certain embodiments, the at least one additional polysaccharide can be different than the primary polysaccharide agent, and the at least one additional polysaccharide can be present in the pre-spray-drying formulation in a weight-to-volume (w/v) concentration from about 0.1% to about 10%, from about 0.1% to about 5%, from about 0.1% to about 2.5%, from about 0.1% to about 0.5%. In certain embodiments, the glass-forming agent present in the pre-spray-drying formulation is sucrose or trehalose and the at least one additional polysaccharide for the formulation is hydroxyethyl starch.

In some embodiments and further to paragraphs [0004]-[0016] above, each layer of the one or more outer coating layers can include one or more of a metallo-organic material, metal oxides, metal alkoxides, and/or aluminum-based coating layer . . . In certain embodiments, each layer of the one or more outer coating layers can include one or more of an aluminum oxide, an aluminum alkoxide (e.g., alucone), silicon dioxide (SiO₂), titanium dioxide (TiO₂), zinc dioxide (ZnO₂) or silicon nitride (Si₃N₄) alone or in a suitable combination composition. In accordance with these embodiments, each coating layer(s) can be about 0.1 nm to about 30.0 nm or about 0.1 nm to about 20.0 nm in thickness. In certain embodiments, the essentially-dry microparticles (e.g., post spray-drying) disclosed herein can include a number of outer coating layers sufficient to delay release or provide a timed-release of the at least one antigen or agent contained in one or more layers of a coated microparticle or from the central or innermost core of the coated microparticle.

In certain embodiments and further to paragraphs [0004]-[0017] above, the one or more coating layer(s) disclosed herein can serve as an adjuvant to enhance an immune response in a subject against the one or more agent(s) or antigen of the agent-or antigen-containing coated microparticles. In some embodiments, the one or more coating layer(s) can include a coating layer or coating layers capable of inducing a rapid immune response in the subject and/or the essentially dried and coated lipid-emulsion having the one or more agent or antigen can be contained in a selected layer or layers and/or the core of the coated microparticles that when the coating layers dissolve and expose the one or more antigen or agent induce a rapid immune response to the one or more agent or antigen in the subject.

In some embodiments and further to paragraphs [0004]-[0018] above, lipid nanoemulsion agent-containing, lipid nanosuspension agent-containing, or antigen containing coated microparticles described herein can be stored without refrigeration at temperatures of room temperature, up to about 50° C., or up to about 60° C., or up to about 70° C. for extended periods of time. In certain embodiments, lipid nanoemulsion agent-containing, lipid nanosuspension agent-containing or antigen-containing coated microparticles described herein can be stored without refrigeration up to room temperature, up to about 50° C. or up to about 60° C. or up to about 70° C. for up to about a day, about two or more days, about a week, about a couple of weeks, about a few weeks, about a month, about 2 months, about 3 months, up to about 4 months, up to about 6 month, up to about 9 months, up to about 12 months, up to about 15 months, up to about 18 months, up to about 24 months or longer without negative effects on the coated microparticles (e.g. degradation, loss of efficacy, loss of immunogenicity, reduced delivery of the one or more agents or antigens) or their encased nanoemulsions or nanosuspensions (e.g., degradation, coalescence of nanoemulsions, agglomeration of nanosuspensions).

Other embodiments and further to paragraphs [0004]-[0019] above, provide for combination compositions or formulations including a plurality of coated lipid emulsion agent-containing or antigen-containing microparticles described herein. In accordance with these embodiments, these combination compositions or formulations can include mixtures of different coated microparticles containing one or more agent for treating or preventing a single health condition or multiple health conditions (e.g., pathogenic agent infections or prevention of pathogenic agent infections) and further include at least one pharmaceutically acceptable excipient to make a pharmaceutically acceptable composition or formulation. In other embodiments, combination compositions can include at least one representative microparticle-containing pharmaceutical composition mixed with a standard or known composition or formulation to treat, reduce onset of, or prevent a health condition (e.g., infection, cancer or other condition).

In some embodiments and further to paragraphs [0004]-[0020] above, formulations disclosed herein can be part of a single-administration formulation including a prime dose and at least one boost dose of at least one agent or at least one antigen. In accordance with these embodiments, the prime and at least one boost dose of the at least one antigen or at least one agent can be in the same coated microparticle, or in separate coated microparticles. When in separate particles, the priming dose of the at least one agent or at least one antigen can be sequestered in a microparticle while the at least one boost dose can be in separate microparticle. In certain embodiments, release of the priming dose after delivery to a subject can be immediate or delayed depending on the number of coating layers coating the lipid-emulsion coated agent-containing or antigen-containing microparticle priming dose (if not on the outermost or near outermost coating layer) and release of the at least one boost can be a short time, days, or several months after the initial dose of agent or antigen containing coated microparticles in the subject.

In some embodiments and further to paragraphs [0004]-[0021] above, ALD-coated formulations containing the lipid-emulsion agent-containing or antigen-containing coated microparticles can be part of a single administration formulation having at least two different agents or two different antigens capable of eliciting an immune response or other treatment response to two or more different antigens or agents. In accordance with these embodiments, the two or more different antigens or different agents can be included in the same or separate coated microparticles.

In other embodiments and further to paragraphs [0004]-[0022] above, ALD-coated lipid-emulsion agent-containing or antigen-containing microparticle compositions can further include a standard vaccine composition (e.g., mRNA, live, attenuated virus,) with a plurality of ALD coated lipid-emulsion agent-containing or antigen-containing microparticles described, where at least one of the at least one agent or at least one antigen elicits a boost immune response to the standard vaccine composition.

Other embodiments and further to paragraphs [0004]-[0023] above, provide for methods for eliciting a response in a subject, where the method can include administering an ALD-coated microparticle-containing formulation described herein to the subject. In accordance with these embodiments, the formulation can be administered by any method known in the art. In other embodiments, the formulation can induce a response (e.g., an immune response) in the subject. The immune response induced by the composition can be prophylactic or therapeutic depending on the at least one antigen or at least one agent.

Yet other embodiments and further to paragraphs [0004]-[0024] above, provide for kits that can include at least one ALD-coated lipid-emulsion agent-or antigen-containing microparticles or components for creating the same described herein. In certain embodiments, kits can further include at least one container and/or instructions for making or using coated formulations disclosed herein.

### BRIEF DESCRIPTION OF THE FIGURES

The accompanying drawings are incorporated into and form a non-limiting part of the specification to illustrate several examples of the present disclosure.

FIG. 1 is a representative table indicating water content of spray-dried samples with varying polysaccharide concentrations according to some embodiments of the present disclosure.

FIG. 2 is a plot of spray-drying versus freezing of various samples in increasing concentrations of a representative salt in order to analyze droplet diameters of emulsions under the various conditions according to some embodiments of the present disclosure.

FIGS. 3A-3B are a representative graph of a tested salt concentration versus emulsion droplet size diameter distribution (a) and a representative plot of the tested salt concentration versus hydrodynamic diameter of microparticles (b) disclosed herein according to some embodiments of the present disclosure.

FIG. 4 is a representative plot illustrating coated versus uncoated lipid emulsion microparticles assessing range of hydrodynamic radius of incorporated lipid nanodroplets over time according to some embodiments of the present disclosure.

FIG. 5 is a representative plot assessing effects of various disaccharide concentrations of spray-dried versus not spray-dried microparticles on maintenance of emulsified nanodroplet size below 300 nm after reconstitution according to some embodiments of the present disclosure.

FIG. 6 is a representative plot assessing effects of various disaccharide concentrations in the presence or absences of an additional disaccharide (e.g., hydroxyethyl starch, HES) of spray-dried versus not spray-dried microparticles on maintenance of emulsified nanodroplet size below 300 nm after reconstitution according to some embodiments of the present disclosure.

FIG. 7 is a representative plot assessing effects of various disaccharide concentrations of spray-dried versus not spray-dried microparticles on maintenance of emulsified nanodroplet size below 300 nm after reconstitution and assessment of effect of ionic strength effect on size distribution according to some embodiments of the present disclosure.

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some embodiments, well known methods or components have not been included in the description.

Vaccine formulations including nanoemulsions of lipids can be thermally unstable and typically must be kept refrigerated to reduce degradation, to maintain immunogenic activity or integrity of the agent(s) or antigen(s) and create reliable deliverables. These issues can complicate vaccine and therapeutic distribution in times of a pandemic, extreme catastrophic events, to underserved and to remote areas for example that could have unreliable electricity to keep thermally unstable agents, antigens and/or vaccines under continuously cold or cool temperatures. Embodiments disclosed herein solve these issues by spray drying nanoemulsified lipids to imbed these agents, antigens and/or vaccines within glassy matrices that limit molecular mobility.

Embodiments disclosed herein and further to paragraph above, provide for novel compositions and methods for making and using thermostable agents, antigens and/or vaccines within nanoemulsified lipid-containing glassy microparticle formulations. In certain embodiments, compositions and methods are disclosed for adapting nanoemulsions of agent-or antigen-containing lipid formulations with improved stability for prolonged storage, transport and/or retaining efficacy of the agents, antigens, and/or immunogenicity. In other embodiments, nanoemulsions of antigen-or agent-containing lipid formulations can be spray-dried to embed within glassy matrices or microparticles to improve stability and compatibility alone or with other agents by limiting molecular mobility. In other embodiments, these essentially dried stabilized nanoemulsions of antigen-or agent-containing lipid formulations can be coated by one or more coating layer to produce stabilized nanoemulsions of lipid-containing agents. In accordance with these embodiments, coatings can include but are not limited to atomic layer deposition (ALD) coatings.

In certain embodiments and further to paragraphs [0035]-[0036] above, spray-dried nanoemulsified antigen-or agent-containing lipid formulations disclosed herein can form essentially dried microparticulate powders where these microparticles include essentially dried nanoemulsified antigen-or agent-containing lipid formulations can be introduced to a fluidized bed atomic layer deposition (ALD) reactor to apply one or more coating layers where each layer of the one or more coating layers can include one or more of a metallo-organic material, metal oxides, metal alkoxides, and/or aluminum-based coating layer. In certain embodiments, the coating layer can include, but is not limited to, aluminum oxide (Al₂O₃), an aluminum alkoxide, silicon dioxide (SiO₂), titanium dioxide (TiO₂), and silicon nitride (Si₃N₄). It is noted herein that these processes do not include solvents and are designed to apply a metal-containing formulation to coat thermostabilized antigen or agents with precision for timed-release of a target antigen or agent to treat a condition in a subject. In accordance with these embodiments, combinations of spray-drying of the nanoemulsified antigen-or agent-containing lipid formulations and ALD-coating increases the thermostability of the nanoemulsified lipid formulations and can create timed-release of the agent or antigen. For example, these coated essentially dry nanoemulsified lipid formulations have improved stability at room temperature storage or higher temperature storage, up to about 60-70° C.

In certain embodiments and further to paragraphs [0035]-[0037] above, the coated adjuvant-, antigen-or agent-containing lipid formulations (e.g., coated lipid nanoparticles) can result in increased stability and/or compatibility of an antigen or other agent of the coated and stabilized antigen-or agent-containing lipid nanoemulsions to provide for timed-release delivery of the antigen or agent contained within the coated antigen-or agent-containing lipid microparticles, increased compatibility between agents (if normally incompatible to combine) if more than one agent or antigen is to be contained within the coated particles, and reduced concentrations of agents and/or antigens are needed to treat, reduce onset of, or prevent a health condition.

In some embodiments and further to paragraphs [0035]-[0038] above, primary and at least one boost dose of ALD coated antigen-or agent-containing lipid glassy microparticles (e.g., coated lipid nanoparticles) can be administered to a subject in a single administration of these coated particles. In other embodiments, ALD coated antigen-or agent-containing lipid glassy microparticles (e.g., coated lipid nanoparticles) disclosed herein can include antigens (e.g., immunogenic antigens) or agents against two or more pathogens or other essentially dried agent-or antigen-containing nanoemulsion formulations in the same or in separate coated microparticles.

In certain embodiments and further to paragraphs [0035]-[0039] above, compositions and methods disclosed herein describe critical formulation parameters and methods for spray-drying of emulsified adjuvants, antigens or agents using an oil-based nanoemulsion system (e.g., triterpenoid such as squalene) for developing thermostable emulsified adjuvant, antigen, or agent-containing formulations for further stabilization of the adjuvants, antigens or agents. In certain embodiments, much or essentially all of bulk liquids or water can be removed from these formulations to attain a formulation with residual moisture of less than about 5.0% w/v, less than about 2.5% w/v, less than about 1.0% w/v, less than about 0.5% w/v to reduce or restrict molecular mobility of an emulsified lipid adjuvant-, antigen-or agent-containing composition disclosed herein. In accordance with these embodiments, an emulsified adjuvant-, antigen-, or agent-containing composition having reduced water content has dramatically reduced or restricted molecular mobility leading to reduced collision frequencies and/or lower rates of droplet coalescence. In certain embodiments, reducing collision frequency by reducing water or moisture content of an adjuvant-, agent-or antigen-containing lipid emulsion composition or formulation can lead to stability at typically adverse temperatures, such as room temperature up to about 60° C., 70° C., or higher. In other embodiments, reduced water or moisture content of these compositions and stabilization at increased temperatures can permit ALD coating of these reduced moisture-containing adjuvant-, agent-or antigen-containing lipid emulsion compositions without degradation of the adjuvant, agent and/or antigen embedded within these formulations.

In some embodiments and further to paragraphs [0035]-[0040] above, compositions of use herein are disclosed for creating a reduced-moisture or reduced water-containing adjuvant-, antigen-or agent-containing lipid emulsion formulation, a stabilized core of these compositions by use of spray-drying in also described. In accordance with these embodiments, these compositions can contain, but are not limited to, one or more salt, one or more glass-forming agents such as one or more polysaccharide or disaccharide (e.g., trehalose, sucrose or similar) and a high molecular weight glass-forming agent for example, hydroxyethyl starch, polyvinylpyrrolidone, dextran, carboxymethyl cellulose, human serum albumin, bovine serum albumin, other serum albumin, or the like. In accordance with these embodiments, in liquid formulations prior to spray-drying salt concentrations can be about 0.1 mM to about 250.0 mM (or about 10.0 mM to about 200.0 mM); glass-forming agents/polysaccharide concentrations can be about 2.5% to about 30.0% (or about 7.50% to about 25.0% or about 7.50% to about 20.0%) and a high molecular weight agent such as an additional glass-forming agent concentration can be about 0.1% to about 10.0% or about 1.0% to about 5.0%. In certain embodiments, a formulation prior to spray-drying disclosed herein contains about 0.1% to about 10.0% or about 1.0% to about 5.0% hydroxyethyl starch, about 2.5% to about 30.0% (or about 7.50% to about 25.0% or about 7.50% to about 20.0%) sucrose and/or about 2.5% to about 30.0% (or about 7.50% to about 25.0% or about 7.50% to about 20.0%) trehalose. In some embodiments, surprisingly, it was discovered that even though adjuvant, agent or antigen-containing lipid emulsions could be or were unstable in liquid solutions, when formulated at the same high salt concentrations found during spray drying (e.g., about 50.0 mM to about 1000.0 mM or about 150.0 mM to about 640.0 mM), salt-induced instabilities of these emulsions were reduced or eliminated by co-formulating the adjuvant-, agent-or antigen-containing lipid emulsions with at least one glass-forming polysaccharide or disaccharide (e.g., hydroxyl starch, sucrose, trehalose or other comparable polysaccharide). In yet other embodiments, instabilities could be further reduced or eliminated if the one or more polysaccharide agents were combined with the adjuvant-, agent-or antigen-containing lipid emulsion formulation prior to spray-drying in the presence of solutions when the one or polysaccharide agents were present in high concentrations (e.g., 0.5 wt % to about 25.0 wt %, or about 2.0 wt % to about 15.0 wt %), where the combination of elevated polysaccharide concentrations and rapid (e.g. about a few seconds to sub-second timing) spray-drying times combined to reduce both droplet-droplet collision frequencies and the time available for these collisions to result in undesirable coalescence or agglomeration of the lipid-containing emulsions or suspensions, leading to stabilized spray-dried formulations. In certain embodiments, quick spray-drying while introducing elevated polysaccharide concentrations to these formulations creates a microparticle core (e.g., a powder) coatable by ALD or similar system for applying a metal-containing coating (e.g., applying a metal oxide agent, metal alkoxide, metalloorganic material or the like). In some embodiments, surprisingly, it was discovered that even though adjuvant-, agent-or antigen-containing lipid emulsions could be or were unstable in liquid solutions, when formulated at the same high salt concentrations found during drying (e.g., 50 mM to about 1000 mM or about 100 mM to about 500 mM), salt-induced instabilities of these emulsions were reduced or eliminated by co-formulating the lipid emulsions with at least one glass-forming polysaccharide, for example, sucrose, trehalose, hydroxyethyl starch or other comparable polysaccharide. In some embodiments, salts of use in liquid formulations disclosed herein prior to spray-drying can include but are not limited to sodium citrate, sodium chloride, sodium phosphate, calcium chloride, potassium chloride, and potassium phosphate and the like or a combination thereof. In some embodiments, the salt includes sodium citrate.

In yet other embodiments and further to paragraphs [0035]-[0041] above, instabilities of adjuvants, agents and/or antigens disclosed herein can be further reduced or eliminated if the one or more polysaccharide agents were rapidly introduced and/or combined with the lipid-emulsion adjuvant-, agent-or antigen-containing formulation and further spray-dried in the presence of solutions containing high concentrations of these one or more polysaccharides (e.g., 2.5% to about 30.0%). In accordance with these embodiments, these combinations of elevated polysaccharide concentrations and rapid (e.g., about 100 milliseconds to about 1.0 second) spray-drying times reduce both droplet-droplet collision frequencies and the time available for these collisions to result in undesirable coalescence or agglomeration of the lipid-containing emulsions resulting in stabilized spray-dried formulations. In certain embodiments, rapid spray-drying while introducing elevated polysaccharide concentrations to these formulations creates a coatable microparticle core (e.g., a coatable powder) to which molecular coating layers can be applied by atomic layer deposition (ALD) or similar system for coating (e.g., applying a metalloorganic agent, metal oxide agent or the like) with precision to generate timed-release coated formulations.

As understood by one of skill in the art and further to paragraphs [0035]-[0042] above, salt concentration can determine ionic strength of a formulation. At high ionic strength, charges on the surface of emulsified droplets can be shielded, reducing repulsive inter-droplet repulsive forces, and leading to decreased distances between emulsion droplets that facilitate droplet-droplet interactions that can result in droplet coalescence. During spray-drying, increasing ionic strength can compress the electrical double layers surrounding the droplets and further lead to decreased droplet distances. Non-reducing polysaccharides such as sucrose and trehalose form glassy matrices during spray-drying. High viscosities within these glassy matrices reduce or prevent translational motion of any lipid nanodroplets of nanoparticles contained within the matrix. In certain embodiments, as disclosed herein, weight ratio of lipid to polysaccharide is a critical parameter for emulsion stability during freezing, lyophilization, and spray-drying of these formulations. In accordance with these embodiments, weight ratio of lipid to polysaccharide is assessed and parameters for improved conditions for spray-drying lipid emulsion agent containing formulations are disclosed herein followed by further stability of spray-dried or essentially dried microparticle coating of these essentially dry lipid-cmulsion adjuvant, antigen-and/or agent-containing microparticles contemplated herein.

In certain embodiments and further to paragraphs [0035]-[0043] above, drying rates in spray-drying can be modulated by the addition of high molecular weight drying agents such as hydroxyethyl starch, carboxymethyl cellulose or similar agent high molecular weight agent. In some embodiments, these high molecular weight drying agents (e.g., hydroxyethyl starch) can precipitate as a film on the surface of droplets from lipid emulsions disclosed herein as they dry during spray-drying, and for example, decrease the rate of drying. In accordance with these embodiments, this results in decreased Peclet numbers for any emulsified nano-droplets or nanoparticles (e.g., lipid emulsions) contained within the droplets, effectively giving the nanodroplets sufficient time to diffuse away from the receding drying region, lowering their concentration at the drying front and reducing their propensity to collide and coalesce, and reducing particle destruction. In other embodiments, lipid-based emulsions that have been stabilized by spray-drying to form glassy powders can be further protected from instabilities by coating the powders with metal oxides or other agents disclosed herein, for example using, nanoscopic layers (e.g., molecular layers) of metal formulation coating through atomic layer deposition (ALD). ALD-coating further increases the thermostability of the emulsified formulation for long term storage, but the coating can be dissolved either in vivo or in a salt buffer. These observations were surprising that the agents within the lipid-based emulsions were stabilized and further stabilized to permit ALD-coating. In addition, it was surprisingly found and disclosed herein that lipid emulsions in polysaccharide-containing formulations that had been spray-dried to form essentially dried microparticles could be further introduced to a fluidized bed atomic layer deposition reactor to coat the microparticle surfaces without altering the size distribution of the embedded lipid emulsion contained with a powder. Thus, the size of the microparticles was maintained as well as their syringability preserving their interior nanoemulsion droplet size distribution for later delivery when reconstituting coated microparticles disclosed herein.

In some embodiments and further to paragraphs [0035]-[0044] above, stabilized lipid-emulsion agent-, adjuvant-or antigen-containing powders, microparticles, or glassy matrices disclosed herein can be coated using ALD with one or more coating layers where each layer of the one or more coating layers can include one or more of a metallo-organic material, metal oxides, metal alkoxides, and/or aluminum-based coating layer. In certain embodiments, the coating layer can include, but is not limited to, aluminum oxide (Al₂O₃), an aluminum alkoxide, silicon dioxide (SiO₂), titanium dioxide (TiO₂), and silicon nitride (Si₃N₄). In accordance with these embodiments, combinations of spray-drying of the nanoemulsified lipid formulations and ALD-coating increases the thermostability of the nanoemulsified lipid formulations. For example, these coated essentially dry nanoemulsified lipid formulations have improved stability at room temperature storage or higher temperature storage.

In certain embodiments and further to paragraphs [0035]-[0045] above, ALD coated lipid-emulsion agent, lipid-cmulsion adjuvant, lipid-emulsion antigen-containing powders, microparticles or glassy matrices can be stored, transported, and reconstituted for use to treat, reduce onset, or prevent a medical condition. In accordance with these embodiments, reconstituted coated microparticles maintain size distributions that are syringable and capable of being introduced to a subject by any delivery method known in the art.

In certain embodiments and further to paragraphs [0035]-[0046] above, one or more agent disclosed herein can include at least one immunogenic agent in a lipid emulsion that forms part of a central or innermost microparticle. In accordance with these embodiments, the central or innermost microparticle can include, but is not limited to, at least one essentially dry lipid emulsion-immunogenic agent and at least one glass-forming agent or polysaccharide-containing microparticle, and further, one or more outer coating layers covering or encasing the central or innermost lipid emulsion immunogenic agent-containing glassy microparticle. In accordance with these embodiments and those referenced above, one, two, three, four, five, up to 10, up to 20, up to 30, up to 40, up to 50, up to 100, up to 150, up to 200, up to 250, up to 300, up to 350, up to 400, up to 450 or more coating layers can encase stabilized lipid-emulsion agent(s), lipid-emulsion adjuvants, or antigen-containing powders or glassy matrices where the coating layers are readily dissolvable in a subject once administered to the subject, to expose the one or more agent or antigen to the subject by immediate exposure or timed-release. In other embodiments, the at least one agent, at least one adjuvant and/or the at least one antigen can be stabilized in powder form as disclosed herein and encased in another layer or outer layer of coating agent forming a prime dose of the at least one agent to be exposed prior to dissolution of the microparticles to expose an antigen, adjuvant, and/or agent in an outer layer or expose at least one of an antigen or agent of the inner core. In yet other embodiments, the at least one agent or at least one antigen can include a mixture of agents or separate agents in the inner core from the same or different species or derived from the same or different agent or compound family and then layered on a coated microparticle, or mixed and coated on a coated microparticle, or mixed with at least one outer layer of coating to create coated microparticles for differential exposure of the at least one agent or antigen or the like. An outer layer of coating by the same or different antigen, agent and/or adjuvant can further be coated by additional metal-containing compositions using ALD.

Certain embodiments and further to paragraphs [0035]-[0047] above, methods for making stabilized lipid-emulsion agent(s), adjuvant-and/or antigen-containing coated microparticles, stabilized powders or glassy matrices, the methods including combining at least one lipid emulsion-containing agent/adjuvant/antigen with at least one glass-forming agent or polysaccharide to form a primary liquid composition, rapidly spray-drying the composition to form lipid emulsion agent-containing glassy microparticles, and coating the essentially-dried lipid emulsion agent-containing glassy microparticles with one or more outer coating layers. In some embodiments, a primary liquid composition (prior to spray-drying) can further include rapid introduction of a second glass-forming agent. In accordance with these embodiments, the second glass-forming agent or polysaccharide can include hydroxyethyl starch or similar agent. Further, in accordance with these embodiments, hydroxyethyl starch can precipitate as a film on the surface of droplets of the composition as they dry during spray drying, decreasing the rate of drying and reducing collision of particles thereby maintaining integrity of the particles as they dry to a more stabilized state in preparation for coating. Rapid spray drying as disclosed herein can mean spray-drying in a few milliseconds up to a few seconds. In accordance with these embodiments, particulates and/or essentially dry microparticles disclosed herein are formulated and spray dried such that therapeutic agents of the lipid nanoemulsions of therapeutic agent-containing formulations are not principally located at the surface of the spray dry particle (reduced exposure). It is known that high molecular weight compounds and low solubility compounds have a higher likelihood of ending up at the surface of a spray dried particle, therefore, embodiments disclosed herein address this commonly observed issue by specific additions to the formulation to form an external shell-like layer that is not principally the target therapeutic agent and can, for example, shield the polynucleotides making them more stable under certain conditions. In accordance with these embodiments, particulates and/or essentially dry microparticles disclosed herein can be introduced to an ALD reaction chamber where the particulates and/or essentially dry microparticles flow freely within the chamber for reduced agglomeration and/or aggregation of the particulates and/or essentially dry microparticles. It is noted herein that introduction of lipid-emulsion agent containing formulations directly to an ALD reaction chamber without creating thermostable particulates or essentially dry glassy microparticles as disclosed herein would be an unsuccessful coating protocol; for example, the elevated temperature would degrade the formulation and therapeutic agent or the therapeutic agent would likely stick to one another or the chamber and/or the metal material applied would not layer onto free therapeutic agent (not encased in a protective shell or glassy particle) and/or fully encapsulate the therapeutic agent because there would be no availability of binding groups needed to secure the coating which is created herein using polymer-containing formulations to create the essentially dry microparticles or particulates disclosed herein for long term storage or for further coating. It was not known until the instant disclosure that formulations disclosed herein could create such a barrier or shell to protect polynucleotides from such conditions. It is understood by one of skill in the art that formulations and processes disclosed herein are scalable and readily available for manufacture for creating bulk microparticles for coating, or storage and later coating.

In accordance with these embodiments and further to paragraphs [0035]-[0048] above, the at least one agent, at least one adjuvant and/or at least one antigen can include one or more agent(s) or antigen(s), and/or adjuvants obtain from or part of, for example a viral antigen, a bacterial antigen, a toxin, a prion, yeast, a fragment or subunit thereof, a chemical agent, a small molecule, an anti-cancer agent, an anti-inflammatory agent, an anti-autoimmune agent, a peptide, polynucleotide, or protein thereof or a combination thereof. In some embodiments, the at least one agent, adjuvant and/or antigen can include one or more agent(s), adjuvants, and/or antigen(s) including, but is not limited to, a recombinant peptide, a recombinant protein, a peptide derived from a target protein or pathogen, a synthetic peptide or protein, a polynucleotide encoding a polypeptide, a polynucleotide, a virus-like particle, a live virus, a live, attenuated virus, an inactivated virus, or expressed on the surface of a virus or a bacteriophage or a combination thereof. In yet other embodiments, the at least one agent and/or antigen can include one or more agent(s) and/or antigen(s) represented by a polynucleotide, encoded by a polynucleotide or the like. In accordance with these embodiments, a polynucleotide can include, but is not limited to, single stranded (ss) or double-stranded (ds) DNA such as linear or circular DNA, RNA, mRNA, siRNA, or a chimeric molecule thereof, or other polynucleotide thereof. In certain embodiments, a chimera can include a combination of at least one polynucleotide segment and at least one polypeptide segment, or a combination of polynucleotide segments, and/or polypeptide segments. In some embodiments, the polynucleotide can be mRNA encoding a full length or fragment thereof, for example, of a targeted agent or antibody or receptor molecule thereof (e.g., virus or bacteria).

In certain embodiments and further to paragraphs [0035]-[0049] above, the at least one agent or antigen can include, but is not limited to, at least one pathogen or antigen derived therefrom for example, antigens derived from human papilloma virus (e.g., HPV16, HPV18) or other mammalian papilloma virus, ricin toxin, Bacillus anthracis, Clostridium botulinum, Ebola virus, influenza virus, Corona virus (Covid-19 or other variants, mutants thereof), poliovirus, norovirus, rotavirus, hepatitis C, varicella, herpes simplex, cytomegalovirus, Japanese encephalitis, dengue virus, West Nile virus, Zika virus or other flaviviruses, an alphavirus such as chikungunya, EEEV, WEEV, VEEV or other alphavirus, pneumonia virus spp., Yersinia, Pneumococcus, Salmonella, Clostridium difficile, or a combination thereof. In certain embodiments, the at least one antigen or agent can be a multimeric complex. In other embodiments, the at least one antigen or agent can include a pathogen or antigen derived therefrom capable of infecting a human (e.g., adult, child, toddler, infant or fetus), companion animal, livestock, wild animal, zoo animal, bird, or reptile.

In some embodiments and further to paragraphs [0035]-[0050] above, the at least one glass-forming agent or polysaccharide disclosed herein can include at least one of trehalosc, sucrose, ficoll, dextran, maltotriose, lactose, mannitol and glycine, hydroxyethyl starch, glycine, cyclodextrin, povidone, or the like. In certain embodiments, the at least one glass-forming agent or polysaccharide disclosed herein can include trehalose or sucrose or combination thereof. In certain embodiments, the at least one glass-forming agent or polysaccharide disclosed herein can include hydroxyethyl starch alone or in combination with a second polysaccharide. In accordance with these embodiments, the at least one glass-forming agent or polysaccharide disclosed herein can be present in a primary liquid composition prior to spray-drying in a weight-to-volume (w/v) concentration of from about 0.1% to about 40%, from about 1.0% to about 30.0%, from about 5.0% to about 30.0%, about 5.0% to about 25.0%, about 5.0% to about 20.0%, about 5.0% to about 15.0% or about 20%. In certain embodiment, the at least one glass-forming agent or polysaccharide disclosed herein can be rapidly introduced to the lipid-emulsion agent(s) or antigen and/or adjuvant and further spray-dried to an essentially dried formulation; optionally, in preparation for application of one or more coating layers to the essentially dried microparticles formed using an ALD process as described herein. In yet other embodiments, the glass-forming agent or polysaccharide includes sucrose at a concentration of about 1.0% to about 40.0% or about 5.0% to about 30.0%, or about 10.0% to about 30.0% or about 15.0% to about 30.0% or about 20.0% to about 40.0% or about 20.0%. In certain embodiments, drying of microdroplets containing nanoemulsified lipid-emulsions by spray drying as disclosed herein can occur over a rapid timescale of about 50 milliseconds to about 3 seconds, or about 100 milliseconds to about 1 second, or about 300 milliseconds.

In other embodiments and further to paragraphs [0035]-[0051] above, the glass-forming agent or polysaccharide disclosed herein can include at least one additional polysaccharide agent (or disaccharide agent). In accordance with these embodiments, the at least one additional polysaccharide agent can include, but is not limited to, hydroxyethyl starch (HES), dextran, hetastarch, carboxymethylcellulose, and the like, or a combination thereof. In accordance with these embodiments, the at least one additional polysaccharide can be present in a primary formulation prior to spray-drying at a weight-to-volume (w/v) concentration from about 0.1% to about 40%, from about 1% to about 30%, from about 5% to about 20%, or from about 8% to about 20%. In certain embodiments, the at least one additional polysaccharide agent can be different than a primary or first polysaccharide agent, and the additional polysaccharide agent can be present in a liquid or aqueous pre-spray-drying formulation in a weight-to-volume (w/v) concentration from about 0.1% to about 10.0%, from about 0.1% to about 5.0%, from about 0.1% to about 2.5%, from about 0.1% to about 0.5%. In certain embodiments, a first or primary glass-forming agent present in a pre-spray-drying formulation can be sucrose or trehalose and the at least one additional polysaccharide or disaccharide can be hydroxyethyl starch (HES).

In some embodiments and further to paragraphs [0035]-[0052] above, each layer of the one or more ALD coating layers can include one or more of a metallo-organic material, metal oxides, metal alkoxides, and/or aluminum-based coating layer applied to the essentially dried lipid-emulsion agent-, adjuvant-and/or antigen-containing microparticles. In certain embodiments, each layer of the one or more coating layers can include one or more of an aluminum oxide, an aluminum alkoxide (e.g., aluconc), silicon dioxide (SiO₂), titanium dioxide (TiO₂), Zinc dioxide (ZnO₂) or silicon nitride (Si₃N₄) alone or in a suitable combination or pattern of alternating layering compositions. In accordance with these embodiments, each coating layer(s) can be about 0.1 nm to about 20.0 nm in thickness. In certain embodiments, the essentially-dry microparticles disclosed herein can include a plurality of outer coating layers sufficient to delay release or provide a timed-release of the at least one antigen, at least one adjuvant, and/or agent contained in one or more layers of a coated microparticle or from the central or innermost core of the coated microparticles. In certain embodiments, one, two, three, four, five, up to 10, up to 20, up to 30, up to 40, up to 50, up to 100, up to 150, up to 200, up to 250, up to 300, up to 350, up to 400, up to 450 or more coating layers can encase stabilized lipid-emulsion agent-, adjuvant-and/or antigen-containing powders or glassy matrices or microparticles disclosed herein where the coating layers are readily dissolvable in a subject once administered to the subject by immediate or timed-release, to expose the one or more agent, adjuvant and/or antigen to the subject.

In certain embodiments and further to paragraphs [0035]-[0053] above, the one or more ALD coating layer(s) disclosed herein can serve as an adjuvant to enhance an immune response in a subject against the one or more agent(s) or antigen of the agent-or antigen-containing ALD coated microparticles. In some embodiments, the one or more coating layer(s) can include a coating layer or coating layers capable of inducing a rapid immune response in the subject upon exposure. In other embodiments, coated essentially dried lipid-emulsion having the one or more agent or antigen can be contained in a selected layer or layers and/or the core of the coated microparticles such that when the coating layers dissolve and expose the one or more antigen and/or agent a rapid immune response is induced in the subject to the one or more agent or antigen in the subject. In accordance with these embodiments, the immune response is enhanced compared to delivery of the antigen and/or agent without spray-drying and/or without coating of stabilized microparticles disclosed herein.

In some embodiments and further to paragraphs [0035]-[0054] above, lipid emulsion agent-containing and/or antigen-containing coated microparticles described herein can be stored without refrigeration temperatures at room temperature, up to about 50° C., or up to about 60° C., or up to about 70° C. for extended periods of time. In certain embodiments, lipid emulsion agent-containing and/or antigen containing coated microparticles described herein can be stored without refrigeration up to room temperature, up to about 50° C., or up to about 60° C., or up to about 70° C. for up to about a day, about two or more days, about a week, about a couple of weeks, about a few weeks, about several weeks, about 1 month, about 2 months, about 3 months, up to about 4 months, up to about 6 month, up to about 9 months, up to about 12 months, up to about 15 months, up to about 18 months, up to about 24 months or longer without negative effects on the coated microparticles or the microparticles stabilized but not coated (e.g. degradation, loss of efficacy, loss of immunogenicity, reduced delivery of the one or more agents or antigens).

Other embodiments and further to paragraphs [0035]-[0055] above, provide for combination compositions or formulations including a plurality of coated lipid emulsion agent-containing, adjuvant-containing, and/or antigen-containing microparticles described herein. In accordance with these embodiments, these combination compositions or formulations can include mixtures of different coated microparticles containing one or more agent or antigen for treating, reducing onset of, or preventing a single health condition or multiple health conditions (e.g., pathogenic agent infections or prevention of pathogenic agent infections, cancer, inflammation etc.) and further include at least one pharmaceutically acceptable excipient to make a pharmaceutically acceptable composition or formulation. In other embodiments, combination compositions can include at least one representative microparticle-containing pharmaceutical composition as disclosed herein mixed with a standard or known composition or formulation to treat, reduce onset of, or prevent a health condition. In certain embodiments, the standard formulation can include a standard vaccine against a pathogen or an anti-cancer agent or the like. In some embodiments, ALD coated microparticles disclosed herein can be used as one or more boost doses to a standard treatment directed to treat, prevent, or reduce onset of a health condition.

In some embodiments and further to paragraphs [0035]-[0056] above, formulations disclosed herein are part of a single-administration formulation including a prime dose and at least one boost dose of at least one agent or at least one antigen sequestered within coated microparticles disclosed herein. In accordance with these embodiments, the prime and at least one boost dose of the at least one antigen or at least one agent can be in the same coated microparticle, or in separate coated microparticles. When in separate particles, the priming dose of the at least one agent or at least one antigen can be sequestered in a microparticle while the at least one boost dose can be in separate microparticle. In certain embodiments, release of a priming dose after delivery to a subject can be immediate or delayed depending on the number of coating layers coating the lipid-emulsion coated microparticle priming dose (if not on the outermost or near outermost coating layer) and the at least one boost can be exposed to the subject a short time, minutes, hours, days, weeks, or several months later. In certain embodiments, compositions can be mixed where one composition contains stabilized coated or uncoated stabilized lipid emulsion agent or antigen-containing microparticles alone or in combinations of different agents or antigens targeting the same or different pathogens or health condition or mixtures with other known agents or formulations.

In some embodiments and further to paragraphs [0035]-[0057] above, formulations containing the lipid-emulsion agent-containing or antigen-containing coated microparticles can be part of a single administration formulation having at least two different agents or two different antigens capable of eliciting an immune response to two or more different antigens or agents. In accordance with these embodiments, the two or more different antigens or different agents can be included in the same or separate coated microparticles in the same or different coated particle layers of the microparticle (e.g., in the core and at least in one outer layer). Precision coating layers can be applied by ALD in order to create timed-release of the one or more agent at predetermined periods once administered to a subject.

In other embodiments and further to paragraphs [0035]-[0058] above, thermostable lipid-emulsion agent-containing or antigen-containing microparticle compositions can include a standard vaccine composition (e.g., mRNA, live, attenuated virus,) and a plurality of coated or uncoated lipid-emulsion agent-containing or antigen-containing microparticles described, where at least one of the at least one agent elicits a boost immune response to the standard vaccine composition.

Other embodiments and further to paragraphs [0035]-[0059] above, provide for methods for eliciting a response in a subject, where the method can include administering a coated microparticle of a lipid-emulsion containing agent, adjuvant and/or antigen formulation described herein to the subject to treat, prevent or ameliorate a health condition. In accordance with these embodiments, the formulation can be administered by any method known in the art. In other embodiments, the formulation can induce a response (e.g., an immune response, an anti-inflammatory response, an anti-cancer response or other response) in the subject. In accordance with these embodiments, an immune response induced by the formulation or composition can be prophylactic or therapeutic depending on the at least one antigen and/or the at least one agent sequestered in one or more coated or uncoated microparticles of the lipid-emulsion containing agent or antigen formulation.

In certain embodiments and further to paragraphs [0035]-[0060] above, coated or uncoated lipid-emulsion agent-containing or antigen-containing microparticles and formulations or compositions thereof disclosed herein can use less antigen or agent than used to formulate current vaccines against an immunogen or other therapeutic agent (e.g., cost saving), and provide enhanced efficacy after a single administration. In other embodiments, coated or uncoated lipid-emulsion agent-containing or antigen-containing microparticle compositions or formulations provide for thermostable formulations that eliminate and/or reduce refrigeration requirements (e.g., cold chain refrigeration requirements), limit the concentration of adverse effects of agents (e.g., aluminum, excess therapeutic) administered to a subject, and increase lipid-emulsion agent or antigen compatibility. In certain embodiments, compositions and methods disclosed herein are applicable to a variety of potential agents or antigens, including, but not limited to, polynucleotides, chimeras, carbohydrates, polypeptides, recombinant peptides or protein immunogens, virus-like particles (VLPs), and inactivated or attenuated pathogens (e.g., viruses), bacteriophage conjugated or associated with various antigens and/or agents.

Some embodiments disclosed herein and further to paragraphs [0035]-[0061] above, relate to methods of dehydration or drying and formulation parameters, where these parameters can be adjusted in order to control nucleation rates, glass transition temperatures, and other material properties of the lipid-emulsion agent-containing or antigen-containing microparticles. In certain embodiments, dehydration or drying of agents, antigens or other agents can occur, for example, by spray-drying, and/or spray-freeze-drying or other equivalent procedure to essentially dry a lipid-emulsion agent-containing or antigen-containing microparticle to facilitate coating as disclosed herein. In accordance with these embodiments, the lipid-emulsion agent-containing, adjuvant-containing and/or antigen-containing microparticle can be encased with one or more coating layers to produce lipid-emulsion agent-containing, adjuvant-containing, or antigen-containing coated microparticle. In other embodiments, lipid-emulsion agent-containing or antigen-containing microparticle having one or more antigen or agent directed to one or more pathogens, one or more targeted molecule, one or more small molecule either in the same microparticle or in separate microparticles, can be combined into an aqueous solution, reconstituted. These lipid-emulsion agent-containing or antigen-containing microparticle formulations described herein are thermostable and can be generated against any pathogenic agent or for any therapeutic agent for treatment, amelioration, or prevention of a health condition. In certain embodiments, lipid-emulsion agent-containing or antigen-containing microparticles can be generated to be used against any pathogenic organism or as a therapeutic to treat any condition.

In certain embodiments and further to paragraphs [0035]-[0062] above, a pathogenic virus can be, for example, any pathogenic virus. In accordance with these embodiments, a pathogenic virus can include, but is not limited to, a papovavirus (e.g., papillomaviruses, including human papilloma virus (HPV)), a herpesvirus (e.g., herpes simplex virus, varicella-zoster virus, bovine herpesvirus-1, cytomegalovirus), a poxvirus (e.g., smallpox virus), a reovirus (e.g., rotavirus), a parvovirus (e.g., parvovirus B19, canine parvovirus), a picornavirus (e.g., poliovirus, hepatitis A), a togavirus (e.g., rubella virus), a hepadnavirus (e.g., hepatitis B virus), a flavivirus (e.g., dengue virus, hepatitis C virus, West Nile virus, yellow fever virus, Zika virus, Japanese encephalitis virus or other flavivirus), an orthomyxovirus (e.g., influenza A virus, influenza B virus, influenza C virus), a paramyxovirus (e.g., measles virus, mumps virus, respiratory syncytial virus, canine distemper virus, parainfluenza viruses), a rhabdovirus (e.g., rabies virus), a filovirus (e.g., Ebola virus), an alphavirus (e.g., chikungunya or WEEV, EEEV or VEEV, or other alphavirus) or a coronavirus, SARS or the like or mutants thereof or combinations thereof. In accordance with these embodiments, agents or antigens derived therefrom can be part of a microparticle disclosed herein and further include a pharmaceutically acceptable agent.

In other embodiments and further to paragraphs [0035]-[0062] above, a pathogenic agent or antigen derived from a pathogenic agent can be a bacterium or a toxin of a bacterium or toxoid agent. In accordance with these embodiments a pathogenic agent or antigen derived from a pathogenic agent can include, but is not limited to, Pasteurella haemolytica, Clostridium difficile, Clostridium haemolyticum, Clostridium tetani, Corynebacterium diphtheria, Neorickettsia resticii, Streptococcus equi equi, Streptococcus pneumoniae, Salmonella spp., Chlamydia trachomatis, Bacillus anthracis, Yersinia spp., and Clostridium botulinum or other pathogenic bacteria or a combination thereof. In accordance with these embodiments, agents or antigens derived therefrom can be part of a microparticle disclosed herein and further include a pharmaceutically acceptable agent.

In some embodiments and further to paragraphs [0035]-[0062] above, a pathogenic agent can be a fungus. In accordance with these embodiments, a pathogenic fungus can include but is not limited to, Cryptococcus spp. (e.g., neoformans and gatti), Aspergillus spp. (e.g., fumigatus), Blastomyces spp. (e.g., dermatitidis), Candida albicans, Paracoccidioides spp. (e.g., brasiliensis), Sporothrix spp. (e.g., schenkii and brasiliensis), Histoplasma capsulatum, Pneumocystis jirovecii and Coccidioides immitis, or other pathogenic fungus or combinations thereof. In accordance with these embodiments, agents or antigens derived therefrom can be part of a microparticle disclosed herein and further include a pharmaceutically acceptable agent.

In yet other embodiments and further to paragraphs [0035]-[0062] above, a pathogenic agent can be a toxin. In accordance with these embodiments, a toxin can include but is not limited to, anthracis toxin, ricin toxin or botulinum toxin or other toxin. In accordance with these embodiments, agents or antigens derived therefrom can be part of a microparticle disclosed herein and further include a pharmaceutically acceptable agent.

In yet other embodiments and further to paragraphs [0035]-[0066] above, the one or more antigens or agents can include DNA or mRNA encoding one or more antigens derived from pathogenic agents, including, but not limited to DNA or mRNA encoding for viral capsid proteins or subunits thereof, virus DNA or mRNA encoding for virus-like particles, DNA or mRNA encoding for viral spike proteins, DNA or mRNA encoding for viral enzymes and DNA or mRNA encoding for viral structural proteins . . .

In some embodiments and further to paragraphs [0035]-[0067] above, lipid emulsion agent-containing particles described herein can be used to manufacture one or more microparticle-containing formulation for use as vaccines for any animals. In certain embodiments, the animal can be a household pet or other companion animal. In some embodiments, an animal can include livestock or other farm animal, wild animal, or zoo animal. In certain embodiments, the animal can include a non-human mammal, reptile, or bird. In accordance with these embodiments, the immunogenic composition can be administered, including, but not limited to, to a dog (canine), a cat (feline), a horse (equine), cattle (bovine), a goat (hircine), a sheep (caprine), pig (swine), or poultry (e.g., chicken, turkey, duck, goose).

In certain embodiments and further to paragraphs [0035]-[0062] above, lipid emulsion agent-containing coated microparticles described herein can be used to generate one or more compositions or other agent-containing compositions contemplated herein for administering to a canine to reduce onset of or prevent an infection or treat a condition such as cancer or an inflammatory condition or diabetes or other condition. In accordance with these embodiments, an infection can include, but is not limited to, infections related to canine parvovirus (CPV), canine distemper virus (CDV), canine adenovirus (CAV), rabies, canine parainfluenza virus (CPiV), canine influenza virus, canine corona virus, measles virus, Bordetella bronchiseptica, Leptospira spp., and Borrelia burgdorferi or combinations thereof.

In certain embodiments and further to paragraphs [0035]-[0062] above, lipid emulsion agent-containing coated microparticles described herein can be used to generate one or more compositions or other agent-containing compositions contemplated herein for administering to a feline to reduce onset of or prevent an infection or treat a condition such as cancer or an inflammatory condition or diabetes or other condition. In some embodiments, lipid emulsion agent-containing coated microparticles described herein can be used to generate coated-microparticle containing formulations of use to treat, reduce the risk of onset or prevent an infection or treat an infection in a feline, including but not limited to, immunogenic compositions directed to feline herpesvirus 1 (FHVI), feline calicivirus (FCV), feline panleukopenia virus (FPV), rabies, feline leukemia virus (FELV), feline immunodeficiency virus, virulent systemic feline calicivirus, Chlamydophila felis, Pasteurella haemolytica, and Bordetella bronchiseptica or combinations thereof.

In certain embodiments and further to paragraphs [0035]-[0062] above, lipid emulsion agent-containing coated microparticles described herein can be used to generate one or more compositions or other agent-containing compositions contemplated herein for administering to a horse to reduce onset of or prevent an infection or treat a condition such as cancer or an inflammatory condition or diabetes or other condition. In other embodiments, lipid emulsion agent-containing coated microparticles described herein can be used to generate coated-microparticle containing formulations of use to treat, reduce the risk of onset or prevent an infection or treat an infection in a horse including but not limited to, immunogenic compositions directed to Eastern equine encephalomyelitis virus, Western equine encephalomyelitis virus, Venezuelan equine encephalomyelitis virus, bovine papillomavirus, rabies virus, Clostridium tetani, West Nile virus, equine influenza virus, Potomac fever (Neorickettsia risticii), Streptococcus equi equi, and rhinopneumonitis (equine herpesevirus type 1) or combinations thereof.

In certain embodiments and further to paragraphs [0035]-[0062] above, lipid emulsion agent-containing coated microparticles described herein can be used to generate one or more compositions or other agent-containing compositions contemplated herein for administering to a bovine to reduce onset of or prevent an infection or treat a condition such as cancer or an inflammatory condition or diabetes or other condition. In other embodiments, lipid emulsion agent-containing coated microparticles described herein can be used to generate coated-microparticle containing formulations of use to treat, reduce the risk of onset or prevent an infection or treat an infection in a bovine, including but not limited to, immunogenic compositions directed to bovine rhinotracheitis (IBR), parainfluenza type 3 (PI3), bovine virus diarrhea (BVD), bovine respiratory syncytial virus (BRSV), blackleg (Clostridium chauvoei), malignant edema (Clostridium septicum), infectious necrotic hepatitis (Clostridium novyi), enterotoxemia (Clostridium perfringens type C and D), Pasteurella haemolytica, and redwater (Clostridium haemolyticum) or combinations thereof.

In certain embodiments and further to paragraphs [0035]-[0062] above, lipid emulsion agent-containing coated microparticles described herein can be used to generate one or more compositions or other agent-containing compositions contemplated herein for administering to a poultry to reduce onset of or prevent an infection or treat a condition such as cancer or an inflammatory condition or diabetes or other condition. In other embodiments, lipid emulsion agent-containing coated microparticles described herein can be used to generate coated-microparticle containing formulations of use to treat, reduce the risk of onset or prevent an infection or treat an infection in poultry including but not limited to, immunogenic compositions directed to Marek's disease (Marek's disease virus), tenosynovitis (reoviruses), encephalomyelitis (avian encephalomyelitis virus), fowlpox (avipoxviruses), chicken infectious anemia (chicken anemia virus), fowl cholera (Pasteurella multocida), Newcastle/infectious bronchitis (Newcastle disease virus), Riemerella anatipestifer, duck viral hepatitis (duck hepatitis virus), and duck viral enteritis (duck herpesvirus 1) or combinations thereof.

In certain embodiments and further to paragraphs [0035]-[0062] above, lipid emulsion agent-containing coated microparticles described herein can be used to generate one or more compositions or other agent-containing compositions contemplated herein for administering to a human to reduce onset of, treat, and/or prevent an infection or treat a condition such as cancer or an inflammatory condition or diabetes or other health condition. In other embodiments, lipid emulsion agent-containing coated microparticles described herein can be used to generate coated-microparticle containing formulations of use to treat, reduce the risk of onset or prevent an infection in a human. In certain embodiments, lipid emulsion agent-containing coated particles described herein can be used to deliver one or more formulation disclosed herein to a human such as an infant or child or adolescent, young adult, adult or elderly human subject including but not limited to antigens or agents derived therefor for varicella-zoster (chicken pox), diphtheria, Haemophilus influenzae type b (Hib), hepatitis A, hepatitis B, influenza, corona virus, SARS, Ebola virus, measles, mumps, pertussis, polio, pneumococcal disease, rotavirus, rubella, and tetanus. In other embodiments, immunogenic agent-containing particles described herein may be used to deliver one or more immunogenic compositions to a human pre-teen or teen, including but not limited to vaccines for influenza, tetanus, diphtheria, pertussis, human papillomavirus, meningococcal disease, hepatitis B, hepatitis A, polio, measles, mumps, rubella, and varicella-zoster. In yet other embodiments, lipid emulsion agent-containing coated microparticles described herein can be used to deliver one or more immunogenic compositions to a human adult, including but not limited to immunogenic compositions against influenza (e.g., A, B or C), tetanus, diphtheria, pertussis, zoster, pneumococcal disease, meningococcal disease, measles, mumps, rubella, varicella, hepatitis A, hepatitis B, and Haemophilus influenzae type b.

In other embodiments and further to paragraphs [0035]-[0062] above, lipid emulsion agent-containing coated particles described herein can be used to generate compositions of use for administering to a human. In accordance with these embodiments, treatment of a human subject can include, but not limited to, immunogenic compositions against travel-related infections, including but not limited to, hepatitis A, hepatitis B, typhoid fever, paratyphoid fever, meningococcal disease, yellow fever, dengue fever, rabies, Zika virus-related conditions, West Nile virus infection, Chikungunya disease, and Japanese encephalitis. Covid-19 or corona virus infection, or other infection or combinations thereof.

In yet other embodiments and further to paragraphs [0035]-[0062] above, lipid emulsion agent-containing coated microparticles described herein can be used to generate compositions of use for administering to a human, including but not limited to, agents or antigens. In accordance with these embodiments, agents or antigens can be derived from and include, but not be limited to, human papillomavirus (e.g. HPV 16, HPV18, HPV31, HPV45, or HPV 6 or HPV11, or any other HPV type or serotype), herpes simplex virus, smallpox virus, rotavirus, parvovirus B19 vaccine, chikungunya virus, dengue virus (e.g. dengue-1, dengue-2, dengue-3 or dengue-4 or any new dengue strain or variant), norovirus, hepatitis C virus, West Nile virus, Zika virus, respiratory syncytial virus, rabies virus, and Ebola virus, SARS, COVID-19 or other strain or mutant, or the like.

In certain embodiments and further to paragraphs [0035]-[0076] above, lipid emulsion agent-containing coated microparticles described herein can include a single agent dose or two or more doses of a particular agent (e.g., prime and boost doses or just doses of the agent for prolonged and timed-release). In some embodiments, a lipid emulsion agent-containing coated microparticles can include doses for two or more different immunogenic agents. In yet other embodiments, lipid emulsion agent-containing coated microparticles including doses of different agents can be combined into a mixture of lipid emulsion agent-containing coated microparticles. A mixture of lipid emulsion agent-containing coated microparticles can be combined into a single administration. This can result in a reduced number of administrations and reduced need to return to a clinic for a subsequent administration.

In certain embodiments and further to paragraphs [0035]-[0077] above, lipid emulsion agent-or antigen-containing coated particles (e.g., microparticles) described herein include methods disclosed herein that concern controlled, ultra-rapid freezing rates combined with various concentrations of glass-forming agents as indicated herein. In accordance with these embodiments, glass-forming agents can include, but are not limited to, trehalose and sucrose or similar polysaccharide or the like. These agents can be used to generate glass-like or scaffold-like matrices upon these quick spray-drying procedures. In certain embodiments, when the glass-forming agents are dried during a dehydration process (e.g., spray-drying) in the presence of one or more agents, antigens or therapeutic agent, these form powders (lipid emulsion agent or antigen-containing microparticles), containing embedded agents and/or antigens. In this dehydrated state, protein physical and chemical degradation pathways, which require molecular motion, can be inhibited, as are other degradation pathways thereby stabilizing the one or more agent or antigen from degradation. Some advantages of these procedures for creating stabilized coated microparticles include, but are not limited to, reduced need for cold-chain storage, reduced or eliminated need for a separate adjuvant when inducing an immune response to the one or more agent and/or antigen, reduced concentrations of a target agent and/or antigen, and/or prolonged stability at high temperatures.

In other embodiments and further to paragraphs [0035]-[0078] above, one or more adjuvants and/or immune-stimulating agents can be incorporated into the microparticles or layered onto the microparticles. In accordance with these embodiments, the adjuvant or co-adjuvant can be combined with the at least lipid emulsion antigen or agent, and at least one glass-forming agent in a formulation prior to spray-drying or dehydration and/or applied to an outer layer of a coated ALD microparticle disclosed herein.

In some embodiments and further to paragraphs [0035]-[0079] above, one or more antigens or agents contained within a lipid emulsion can be combined with a glass-forming agent to produce an aqueous composition. For example, the composition can then be dehydrated to form agent-containing or antigen-containing glassy microparticles. In other embodiments, adjuvants and/or co-adjuvants can be included in the composition in preparation for dehydration or spray-drying of the combination.

In yet other embodiments and further to paragraphs [0035]-[0080] above, inactivated or attenuated pathogens (e.g. live, attenuated viruses) can be part of a lipid-emulsion containing composition along with at least one polysaccharide and rapidly spray-dried to form glassy microparticles. In accordance with these embodiments, inactivated (or killed) viruses or virus particles, bacteria, fungi, toxins, or other pathogens can be inactivated by any means, for example, chemically or by heat, mechanically and incorporated into thermostable glassy microparticles disclosed herein. Non-limiting examples of inactivated pathogens that can be incorporated into thermostable lipid-emulsion containing glassy microparticles can include inactivated whole-cell pertussis (inactivated Bordetella pertussis), Salmonella typhi, and inactivated polio virus. Live, attenuated viruses or bacteria can similarly be incorporated into the microparticles. Non-limiting examples of attenuated viruses and bacteria that can be incorporated into thermostable glassy microparticles can include measles virus, mumps virus, rubella virus, influenza virus, chicken pox virus, smallpox virus, polio virus, rotavirus, flaviviruses (e.g., dengue virus, yellow fever virus), alphaviruses, filoviruses, rabies virus, typhoid virus, Mycobacterium bovis, Salmonella typhi, and Rickettsia spp. or other known pathogens or the like.

In some embodiments and further to paragraphs [0035]-[0081] above, one or more agents or antigens supplementing formulations disclosed herein, can be included in a thermostable lipid-emulsion agent-containing glassy microparticle that includes at least one antigen or agent. In accordance with these embodiments, supplementing agents contemplated herein can include, but are not limited to, one or more one aluminum-salt adjuvants, one or more buffers, one or more one volatile salts, one or more immunologically-related agents, one or more smoothing excipients, and co-stimulatory agents (e.g., co-immunostimulatory agents). In other embodiments, formulations disclosed herein can include some or all of immunogenic agents such as pathogenic agents, for example, antigens, glass-forming agents, one or more adjuvants, a buffer, immunologically-related agents, and co-stimulatory agents prior to spray-drying in order to create agent-containing glassy microparticles of use in creating ALD-coated thermostable lipid-emulsion agent-containing glassy microparticles described herein. In some embodiments, formulations disclosed herein can be dehydrated, for example, by lyophilization, vacuum-drying, spray drying, or spray-freeze-drying or other system for dehydrating a sample.

In some embodiments and further to paragraphs [0035]-[0082] above, aluminum salts of use to generate an immunogenic thermostable lipid-emulsion agent-containing microparticles can include one or more of aluminum hydroxide, aluminum phosphate and aluminum sulfate, or combinations thereof. In accordance with these embodiments, the aluminum salt can be in the form of an aluminum hydroxide gel (e.g., Alhydrogel®).

In some embodiments and further to paragraphs [0035]-[0083] above, buffers of use to spray-dry or reconstitute microparticles disclosed herein can include, but are not limited to, acetate, succinate, citrate, prolamine, histidine, borate, carbonate or phosphate buffer, or a combination thereof. In certain embodiments, a buffer can include one or more salts of use in forming a lipid emulsion agent-containing glassy microparticle and can include, but is not limited to, one or more of salt including, but not limited to, sodium acetate, sodium succinate, potassium succinate, sodium citrate, sodium phosphate, potassium phosphate and the like or a combination thereof. In certain embodiments, the buffer can include histidine, for example, histidine-HCl. In other embodiments, one or more volatile salts can include, but are not limited to, ammonium acetate, ammonium formate, ammonium carbonate, ammonium bicarbonate, tricthylammonium acetate, tricthylammonium formate, tricthylammonium carbonate, trimethylamine acetate trimethylamine formate, trimethylamine carbonate, pyridinal acetate and pyridinal formate, or combinations thereof. In some embodiments, a volatile salt can include ammonium acetate.

In some embodiments and further to paragraphs [0035]-[0084] above, glass-forming or polysaccharide agents of use herein can include, but are not limited to, one or more of trehalose, sucrose, ficoll, dextran, maltotriose, lactose, mannitol, hydroxyethyl starch, glycine, cyclodextrin, glycine and mannitol, trehalose and sucrose, and povidone, or combinations thereof. In certain embodiments, the glass-forming agent can be sucrose or trehalose. In other embodiment, the trehalose concentration can be present in a weight-to-volume (w/v) concentration from about 0.1% to about 40% in an immunogenic composition prior to dehydration; from about 1% to about 30% w/v; from about 5% to about 20%; or from about 10% to about 20% w/v or about 20% w/v in the composition prior to spray-drying. In another embodiment, the glass-forming agent can be sucrose or trehalose in a concentration from about 10% to about 25%; or about 15% w/v to about 25% w/v in the immunogenic composition prior to dehydration.

In certain embodiments and further to paragraphs [0035]-[0085] above, compositions disclosed herein can include a co-stimulatory agent in order to further boost immune responses if desired to one or more target antigen or one or more agent against a pathogen or other antigen or agent used to treat a health conditions (e.g., cancer). In accordance with these embodiments, a co-immunostimulatory agent can include, but is not limited to, one or more of lipid A, lipid A derivatives, monophosphoryl lipid A, chemical analogues of monophosphoryl Lipid A, CpG containing oligonucleotides, TLR-4 agonists, flagellin, flagellins derived from gram negative bacteria, TLR-5 agonists, fragments of flagellins capable of binding to TLR-5 receptors, saponins, analogues of saponins, QS-21, purified saponin fractions, ISCOMS, and saponin combinations with sterols and lipids, or the like, or any known co-immunostimulatory agent, or combinations thereof.

It is understood in the art that as complexity of a vaccine or antigenic or immunogenic composition increases, long term stability of the antigenic agents typically decreases, for example, when temperatures are elevated such as during storage or transport. In certain embodiments, formulations disclosed herein harboring multiple subunits (e.g., multimeric) can be more complex than single proteins or fragments and can be less stable. For example, formulations disclosed herein having antigens based on multiple capsomere subunit types can be less stable immunogenic compositions than a single subunit type. In certain embodiments, embedding a multimeric unit compositions (e.g., capsomere) within matrices formed during spray-drying, or spray-freeze-drying can enhance or increase thermal stability of the multimeric complex by, for example, by stabilizing tertiary structure of the multimeric unit. Embodiments described herein can include methods and compositions for use in stabilizing any complex pathogenic or other construct for use in forming thermostable microparticles, including, but not limited to, recombinant peptide or protein immunogens, and inactivated or attenuated pathogens or other complex structures having thermal instability when introduced to the process that can be stabilized as disclosed herein.

In certain embodiments and further to paragraphs [0035]-[0087] above, agents used in the thermostable lipid-emulsion agent-containing microparticles of the present disclosure can be of use for prophylactic and/or therapeutic immunogenic compositions. Suitability of agents for use in lipid-emulsion agent-containing microparticles can be tested by reaction with antibodies or monoclonal antibodies which react or recognize conformational epitopes present on the intact target of the agent and based on the agent's ability to elicit the production of neutralizing antiserum. Suitable assays for determining whether neutralizing antibodies are produced are known to those of skill in the art. In this manner, in certain embodiments, it can be verified whether the immunogenic agents of the present disclosure will elicit production of neutralizing antibodies.

In certain embodiments and further to paragraphs [0035]-[0088] above, agents stabilized in microparticles disclosed herein can be coated or sequestered in one or more coating layers. Certain embodiments concern using molecular deposition processes, in methods, compositions, and formulations for generating single-administration, multi-dose, compositions. In some embodiments, microparticles can be generated where the coating or sequestering layers not only serve as adjuvants to induce an immune response, but also provide for separated prime and boost vaccine doses in a single administration to a subject. In some embodiments, thermostabilization can be achieved by a combination of embedding one or more lipid-emulsion agent containing formulations in glassy organic matrices to form one or more stabilized glassy microparticles, and by using molecular deposition processes that enable a wide variety of molecular layers to be applied to the one or more antigen or agent-containing glassy microparticles and encasing the one or more microparticles to obtain encased, or coated microparticles. In accordance with these embodiments, the thickness of these coating or sequestering layers can be controlled to within 1 or 2 angstroms in thickness and can range from about 0.1 nm to about 20.0 nm per layer. In accordance with these embodiments, one or more coating layers can be deposited consecutively upon one another without leaving an ALD reactor or other reactor until fully coated. In certain embodiments, the thickness of these coating or sequestering layers can be from about 5.0 nm to about 25.0 nm. Using a series of alternating, self-limiting surface reactions, these coating layer deposition processes are highly scalable. For example, fluidized bed reactors can be used to allow large bulk quantities of essentially dried lipid emulsion antigen or agent-containing microparticles to be coated with coating layers without agglomeration of the microparticles. This process allows for complete encasement of the one or more microparticles for immediate or timed-release of the one or more antigen or agent.

In some embodiments and further to paragraphs [0035]-[0089] above, molecular deposition techniques can be used to apply nanometer-thick coatings of inorganic, organic, or metallo-organic materials on the surface of essentially dried lipid-emulsion antigen, adjuvant and/or agent-containing microparticles. In certain embodiments, the coating or sequestering layer can be an aluminum-based material including, for example, an aluminum oxide or metal oxide, or metal alkoxide, and/or an aluminum alkoxide (e.g., alucone) or mixture thereof. In accordance with these embodiments, an aluminum-containing material is deposited on or applied to the surface of the one or more microparticles to coat or sequester the one or more microparticles in 1, 2, 3, 4, 5, 6, 7, 8, 9, 10, 11, up to 20, up to 30, up to 50, up to 100, up to 150, up to 200, up to 250, up to 300, up to 350, up to 400 or more layers of the metallo oxide or other metal-based-containing material to form encased or coated microparticles.

In some embodiments and further to paragraphs [0035]-[0090] above, aluminum-based coating layers can be used as a coating applied to essentially dried lipid emulsion microparticles harboring at least one antigen, adjuvant, and/or agent and at least one glass-forming or polysaccharide agent. In certain embodiments, one or more layers of an aluminum-based film (coating) applied by ALD can be formed by coupling trimethyl aluminum to hydroxyl groups of the microparticles, a layer of amine groups can be formed by coupling ethanolamine to the layer of aluminum-containing material, and a second layer of hydroxyl groups can be formed by coupling maleic anhydride to the available amine groups. An ABC-type reaction, for example, can be self-limiting, and can be used to deposit molecular layers of alucone or other metal-containing compositions disclosed herein. Further to these embodiments, hydroxyl groups on the substrate (e.g., microparticles) react with trimethyl aluminum, ethanolamine then reacts, leaving terminal amine groups on the surface, and available maleic anhydride reacts with terminal amine groups, regenerating a surface of hydroxyl groups for a repeat of the coating if desired. This ABC-type molecular deposition process can be repeated to provide additional layers as desired (up to 10, up to 20, up to 100, up to 200, up to 300, up to 400 or more, or any desired coating number in between), and can be used to deliver, for example, 1, 2, 3, 4, 5 or 6 doses of at least one antigen or agent to a subject in a single administration depending on the composition or make-up of the coated or sequestered one or more microparticles. In other embodiments, various chemical substitutes can be used in these coating or sequestering processes (e.g., alternative sources for the aluminum, amine, and/or hydroxyl groups), as would be recognized by one of ordinary skill in the art and based on the present disclosure.

In certain embodiments and further to paragraphs [0035]-[0091] above, a binary reaction sequence can be used to deposit one or more layers of alumina on an essentially dried microparticle. Microparticles can be treated with alternating gas streams containing either trimethyl aluminum or water vapor. In certain embodiments, the number of cycles can be varied to control formation of the coating or sequestering layer on the one or more agent or antigen-containing microparticles. In certain embodiments, it is understood that during these coating processes no solvents are used as the coating of these microparticles by these ALD processes depend on gases. It is further understood that essentially dried agent-, antigen-and/or adjuvant-containing lipid emulsion microparticles disclosed herein introduced to an ALD reactor for coating are introduced for a predetermined time to receive a predetermined number of coating layers on the microparticles depending on what is desired. It is understood that coated microparticles disclosed herein removed from the ALD reactor can be further processed for preparation and use in treating, reducing onset of, or preventing a health condition. In some embodiments, coated microparticles contain coating layers for exposure of the one or more antigen, agent and/or adjuvant at predetermined times (e.g., immediately, within a week, within a month, within 3 months or more or any time in between as desired and pre-selected by the number of coating layers.

In accordance with these embodiments and further to paragraphs [0035]-[0092] above, some advantages of depositing one or more coating layers on essentially dry lipid-emulsion microparticles include, but are not limited to, the coating layers can dissolve slowly or at a pre-determined rate when the microparticles are administered to a subject, thus allowing temporal control of the release of the particle contents (e.g., the one or more agents). Release times can be tailored by adjusting composition(s) of the coating layers and number and/or thickness of molecular layers applied to the immunogenic agent-containing glassy microparticles. In some embodiments, about 10, about 20 to about 200 or more coating or sequestering layers can be used to form the coated or sequestered microparticles of the present disclosure. In some embodiments, release of the antigens or agents from the coated or sequestered immunogenic agent-containing particle's core can occur within hours, to about 1 day, or about 7 days or about 30 days or about 60 days or about 90 days or about 120 days after administration to the subject. In some embodiments, release of the coated or sequestered antigens or agents from the coated microparticles can occur from about 10 days to about 90 days after administration to the subject. In other embodiments, release of the one or more innermost sequestered or coated antigen or agents from the microparticle can occur from about 30 days to about 90 days after administration to the subject. In some embodiments, release of the innermost antigens and/or agents can occur from about 30 days to about 120 days, or about 30 days to about 90 days, or about 30 days to about 90 days, or about 30 days to about 60 days, after administration to the subject. In some embodiments, release of the innermost antigens and/or agents can occur from about 10 days to about 21 days after administration to the subject. In some embodiments, release of the innermost antigens and/or agents can occur from about 14 days to about 21 days after administration to the subject. Further, in some embodiments, release of the innermost antigens and/or agents can occur from about 18 days to about 21 days after administration to the subject.

In certain embodiments and further to paragraphs [0035]-[0093] above, particle size (e.g. microparticle, nanoparticle etc.) of an encased one or more lipid-emulsion agent-or antigen-or other therapeutic agent containing particles or stabilized nanoemulsions of lipid-containing agents is from about 0.1 μm to about 10 μm, or about 1.0 μm to about 5.0 μm. In other embodiments, an encased antigen and/or agent-containing microparticle having multiple layers is less than about 5.0 μm in size. It will be recognized that the elements of the antigen and/or agent-containing glassy microparticles, coating layers, and any additional layers can be provided in concentrations capable of providing a suitable dose of the antigen and/or agent while maintaining an appropriate particle size for case and consistency of delivery to a subject.

In certain embodiments and further to paragraphs [0035]-[0094] above, onc advantage of using one or more aluminum-based materials as coating or sequestering layer(s) is that the aluminum-based materials can also act as an adjuvant but can also be used to reduce the need for supplemental adjuvants at reduced concentrations. In accordance with these embodiments, the aluminum-based coating layers sequestering or surrounding the microparticles expose essentially the same surface chemistries to immunoactive cells as do standard aluminum-based adjuvant particles known in the art. In some embodiments, nanoscopic aluminum-based coating layers layered on lipid-emulsion agent-containing glassy microparticles disclosed herein can be significantly thinner than what is found in particles of conventional vaccines; therefore, total amounts of aluminum per administration can be essentially negligible, enhancing safety and reduced side effects of these agents. In certain embodiments, the aluminum-based coating layer can be sufficiently thin so that the total aluminum concentration per administration of the composition to a subject is less than about 100 μg, less than about 20 μg, less than about 10 μg, less than about 5 μg, or less than about 1 μg or even less.

In some embodiments and further to paragraphs [0035]-[0095] above, coating layers other than aluminum-based coating layers can be used in order to coat or sequester the antigen and/or agent-containing microparticles. In accordance with these embodiments, non-aluminum coating layers including, but not limited to, silicon dioxide (SiO₂), titanium dioxide (TiO₂), Zinc dioxide (ZnO₂) and/or silicon nitride (Si₃N₄) can be used either in combination with aluminum-based coating layers, or alone to the exclusion of aluminum-based coating layers or as mixtures or alternating patterns of layers. With each type of material having different characteristic dissolution times, layers of different materials can be deposited on the lipid-emulsion agent-containing glassy microparticles to vary the temporal release of the agent from the microparticles' core or other layer. In some embodiments, a microparticle can be coated with one or more coating layers of one material, followed by one or more layers of a different material. In accordance with these embodiments, different materials may dissolve more slowly than for example, an aluminum-based coating layer. Using other materials for coating the particles, can reduce the number of aluminum-based layers necessary to provide for a given release time, minimizing the amount of aluminum per dose.

In other embodiments and further to paragraphs [0035]-[0096] above, one or more coating layers can be deposited on an one or more lipid-emulsion agent-, adjuvant-or antigen-or other therapeutic agent containing particles or stabilized nanoemulsions of lipid-containing agents by, for example, atomic layer deposition (ALD, for example any instrumentation capable of atomic layer deposition can be used). ALD includes a thin film deposition technique that is based on the sequential use of a gas phase chemical process. ALD is considered a type of chemical vapor deposition. In certain methods, the majority of ALD reactions use two chemicals, referred to as precursors. These precursors react with the surface of a material one at a time in a sequential, self-limiting, or directed manner. Through the repeated exposure to separate precursors, a thin film can be slowly deposited. Use of ALD to deposit coating layers on immunogenic agent-containing glassy microparticles can be based on sequential, self-limiting reactions and provides for layer thickness control at the Angstrom level and tunable coating layer composition. Examples of ALD procedures of use in methods disclosed herein for depositing coating or sequestering layers on lipid-emulsion agent-containing glassy microparticles can be found in the art now that stabilized microparticles can be provided as discovered and disclosed herein.

In certain embodiment, and further to paragraphs [0035]-[0097] above, the ALD method can be optimized for a particular situation or condition. For example, antigens against a pathogenic organism incorporated into one or more lipid-emulsion agent-or antigen-or other therapeutic agent containing particles or stabilized nanoemulsions of lipid-containing agents can have variable thermostability, and therefore might not be amenable to higher ALD temperatures due to this vulnerability. In certain embodiments, molecular deposition can occur at temperatures at which the at least one agent of the microparticle remains stable. In some embodiments, molecular deposition can occur under vacuum conditions. By performing the molecular deposition under vacuum conditions, coating layers can be applied at reduced temperatures thereby reducing adverse effects of higher temperatures on target immunogenic agents. In certain embodiments, the vacuum conditions required for deposition may be minimal. In some embodiments, the ALD process can occur under a mild vacuum of about 0.1 atmospheres. In other embodiments, ALD can also include incorporation of magnetically-coupled powder mixing devices that can lead to shorter cycle times for deposition of the material by providing uniform distribution of powders and reactants within an ALD reactor.

Embodiments of the present disclosure and further to paragraphs [0035]-[0098] above, provide for thermostable antigen and/or agent-containing lipid-emulsion-containing microparticles and thermostable compositions, where the thermostable immunogenic composition can be produced by formulating the immunogenic agent-containing particles into a pharmaceutical composition. In certain embodiments, these compositions can be used as vaccines.

In one embodiment and further to paragraphs [0035]-[0099] above, a single administration composition can be generated and can include thermostable lipid-emulsion agent-containing microparticles that provide both a prime and boost dose of one or more agents against a pathogen or for other health condition. In some embodiments, prime and boost doses can be included in the same one or more lipid-emulsion agent-or antigen-or other therapeutic agent containing particles or stabilized nanoemulsions of lipid-containing agent particles. In other embodiments, prime and boost doses can be included in separate microparticles. In accordance with these embodiment, whether together or separate, the prime and boost doses are encased in coating layers for delivery to a subject. In certain embodiments, the priming and boost dose of an antigen or agent can form the core of an agent-containing microparticle or the priming and boost dose of an antigen or agent can form separate layers of an one or more lipid-emulsion agent-or antigen-or other therapeutic agent containing particles or stabilized nanoemulsions of agent-containing lipid-emulsion, each layer encased by one or more layers of coating or sequestering materials.

In some embodiments and further to paragraphs [0035]-[0100] above, at least a second outer layer having at least a second agent (e.g. in an aqueous or gelatinous solution) can be adsorbed to or layered on top of the outermost coating layer surrounding the agent-containing microparticles that include a first agent. In certain embodiments, the agent-containing microparticles can be suspended in a solution of a second glass forming agent and a second antigen, and optionally a smoothing excipient, and then freeze-dried or spray dried to produce agent-containing microparticles including an innermost or central first antigen and an outer second antigen. In certain embodiments, the first and second agents and the first and second glass-forming agents can be the same or can be different agents.

In certain embodiments and further to paragraphs [0035]-[0101] above, multi-layered lipid-emulsion agent-containing microparticles disclosed herein can be reconstituted with water or an aqueous buffer composition or other pharmaceutically acceptable solution, for example, to form an immunogenic composition or formulation and subsequently administered to a subject. In one embodiment, an outer, second layer having at least a second immunogenic agent can act as a conventional priming dose. Subsequently, after a predetermined number of days (the number of days can be adjusted by manipulating numbers and/or thicknesses of the coating layers applied), a sufficient amount of the coating dissolves or degrades, allowing release of a second dose (e.g., boost) from the particle core that serves to boost or supplement the priming dose response in the subject.

In other embodiments and further to paragraphs [0035]-[0102] above, priming (e.g. a first antigen) and boost doses of agents against a pathogen or other target can be included in separate agent-containing microparticles. In accordance with these embodiments, the priming dose of the agent can be included in a first thermostable lipid-emulsion agent-containing microparticle (coated with one or more coating layers), or an agent-containing microparticle (no coating). The boost dose of the agent can be included in a second thermostable agent-containing microparticle coated with one or more coating layers as described herein.

As described herein and further to paragraphs [0035]-[0103] above, lipid-emulsion agent-containing microparticles with metalloorganic-based coating layers reduce incompatibilities between the two or more different agents, whether during storage or after being administered to a subject. In other embodiments, lipid-emulsion agent-containing particles can include two or more different agents (e.g., two or more different antigens) in the same microparticle. In these embodiments, the agent-containing microparticles are safeguarded against incompatibilities between the two or more different agents due to their stabilization within the coated glassy matrix, and in certain embodiments due to physical separation of the agents by one or more coating layers.

Protective immunity, allowing a mammal or other animal to resist (delayed onset of symptoms or reduced severity of symptoms or complete elimination of infection), can be due to exposure to the antigen of a pathogen, disease or toxin that otherwise follows contact with the pathogen. Protective immunity is achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes that result in sIGA production by B lymphocytes on mucosa-lined tissues of the body. “Humoral immunity” includes IgG antibodies and IgM antibodies in serum. “Cellular immunity” can be achieved through cytotoxic T lymphocytes or through delayed-type hypersensitivity that involves macrophages and T lymphocytes, as well as other mechanisms involving T cells without a requirement for antibodies. The primary result of protective immunity is the destruction of the pathogen or inhibition of its ability to replicate itself.

Certain embodiments of the present disclosure and further to paragraphs [0035]-above, include methods to elicit an immune response to an agent of coated stabilized microparticle or combination of agents of one or multiple agent-containing microparticles disclosed herein, by administering to the subject a composition including microparticles disclosed herein. The composition including the microparticles can be administered in therapeutically effective amounts. That is, in amounts sufficient to produce a protective immunological response. Generally, the immunogenic or vaccine compositions can be administered in active agent dosages ranging from about 0.001 mg to about 20.0 mg immunogenic agent or about 0.01 mg to about 20.0 mg agent, or about 0.1 mg to about 10.0 mg agent. Single or multiple dosages can be administered in a single administration composition. Where multiple doses of an immunogenic or vaccine composition are administered in a single administration composition, for example, in prime-boost compositions, one of the two doses can be temporally controlled for release at a pre-selected time after administration.

In certain embodiments and further to paragraphs [0035]-[0106] above, administration of therapeutic or vaccine compositions of the present disclosure can be performed using any acceptable means (e.g., parenterally, locally or systemically, including by way of example, oral, intranasal, intravenous, subcutaneous, intradermal, intravaginal, by suppository, intramuscular, and topical administration). In some embodiments, administration of compositions disclosed herein can be affected by factors including a natural route of infection by a particular pathogen. Dosage or dosages administered can depend upon factors including the age, health, weight, kind of concurrent treatment, exposure, if any, and the nature and type of the particular antigen, or agent. Compositions or vaccine composition disclosed herein can be employed in dosage form such as capsules, liquid solutions, suspensions, or elixirs, for oral administration, or sterile liquid formulations such as solutions or suspensions for parenteral or intranasal use.

### Kits

Other embodiments and further to paragraphs [0035]-[0107] above, provide kits of use with the methods (e.g., methods to elicit an immune response in a subject) and compositions described in the present disclosure. In certain embodiments, a kit may contain one more microparticles in a dehydrated form. In certain embodiments, microparticles can also be provided. Different microparticles and/or immunogenic agent-containing glassy microparticles can be mixed together, or separate and provided to a subject. In certain embodiments, different microparticles are provided mixed together or in separated containers for storage and transport. Microparticles can be provided in a known or predetermined quantity and/or at a predetermined ratio so that when the particles are reconstituted, the result is a composition with a known concentration of agent.

In other embodiments and further to paragraphs [0035]-[0108] above, kits are contemplated of use for compositions, and methods described herein. Kits can be portable. In certain embodiments, kits can be used to transport to and be used in remote areas such as military installations or remote villages. The thermostability of the microparticles allows for transport and storage without the need for a cold chain (e.g., refrigeration). This can also be advantageous in healthcare facilities, as it reduces costs associated with cold storage.

In other embodiments and further to paragraphs [0035]-[0109] above, kits can include an appropriate carrier or diluent suitable for reconstituting the dry thermostable particles. In certain embodiments, the carrier or diluent can be a pharmaceutically acceptable aqueous buffer suitable for injection that includes pyrogen-free water and has the ability to resist changes in pH upon addition of an inorganic compound, organic compound, acid, alkali, or dilution with a solvent or diluent.

In some embodiments and further to paragraphs [0035]-[0110] above, kits can include one or more suitable containers, for example, vials, tubes, mini-or microfuge tubes, test tube, flask, bottle, syringe, or other container. Where an additional component or agent is provided, the kit can contain one or more additional containers into which this agent or component may be placed. Kits herein will also typically include a means for containing the microparticles, pharmaceutically acceptable carrier or diluent and any other reagent containers in close confinement for commercial sale. Such containers may include injection or blow-molded plastic containers into which the desired vials are retained.

In yet other embodiments and further to paragraphs [0035]-[0111] above, kits can include coated or uncoated lipid-emulsion agent-containing glassy microparticles and compositions and optionally, instructions for coating the microparticles. In other embodiments, the kit can include an apparatus for performing such coatings on microparticles. In accordance with these embodiments, the kits allow for production of microparticles of use in immunogenic compositions against one or more pathogens.

It will be recognized that the embodiments described herein can be applied to pharmaceutical compositions other than immunogenic compositions. For example, small molecule drugs (e.g., anti-cancer agents) and biologics may be similarly coated as disclosed for microparticles described herein. The coating layers provide for a level of temporally controlled release desirable with certain pharmaceutical agents. The coating layers can serve to reduce exposure to moisture, reducing degradation. These coatings can function to protect water-soluble drug formulations or other moisture sensitive agents from degradation or dissolution until desired exposure to a subject after administration. Further, the embodiments may be used in applications outside of therapeutics. For example, coating layers may be applied to diagnostic markers. The coatings may allow delayed release of the marker, allowing sufficient trafficking/uptake time. This may be beneficial where the marker has a limited half-life. In certain embodiments, compositions, components, formulations, and immunogenic compositions disclosed herein or encapsulated or coated small molecules using layering/coating technologies described herein can be transported, stored, and administered directly to a subject or to an affected bodily region of a subject such as the liver, lymph nodes, stomach, eye, kidney, or brain depending on the ability of the deposited composition to remain in the targeted region.

### EXAMPLES

The materials, methods, and embodiments described herein are further defined in the following Examples. Certain embodiments are defined in the Examples herein. It is understood that these examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Example 1

Emulsion formulation and manufacturing

In one exemplary example, An MF59®-like emulsion was prepared to serve as a model emulsion for stability testing. MF59® is representative oil-in-water emulsion adjuvant and was licensed for use in pandemic and seasonal influenza vaccines in many countries. MF59® is safe and well tolerated in humans. It is noted that the principal component in MF59® is squalene oil, a naturally-occurring oil found in humans, plants and animals. The squalene oil in MF59® is derived from fish oil and is highly purified before it is used. Initially, for the aqueous phase, 0.5 g of polysorbate 80 was dissolved into 94 mL sodium citrate buffer (10 mM, pH 7.0). The oil phase was prepared by dissolving 0.5 g Span 85 into 5 mL (=4.3 g) squalene. The oil phase was added to the aqueous phase, and this mixture was pre-emulsified by using a high shear homogenizer (e.g., The VirTis Company) at 5,000 rpm for 5 minutes to give a primary oil-in-water emulsion. Then, the primary emulsion was processed through an Emulsiflex C5 high pressure homogenizer (e.g., Avestin) with 5 passes at an operating pressure at 7,000-8,000 psi. After 5 passes, the emulsion was collected and filter-sterilized with a 0.2 μm polyether sulfone (PES) filter. The filtered emulsion was stored at about 2-8° C. until use. Measurement of emulsion droplet size and charge by dynamic light scattering (DLS) and zeta potential analysis

In another exemplary method, emulsion droplet hydrodynamic diameters and zeta potentials were measured using an Anton Paar Litesizer 500 (Graz, Austria). Prior to analysis, emulsion samples were diluted 1:100 with water (e.g., MilliQ®). Dynamic light scattering analysis was conducted using back scattering at about 175°. Hydrodynamic diameters are the intensity-weighted mean hydrodynamic sizes and were derived from a cumulative analysis of the measured correlation curve. Zeta potentials were calculated from the electrophoretic mobility using standard equations integrated into the software used for analysis.

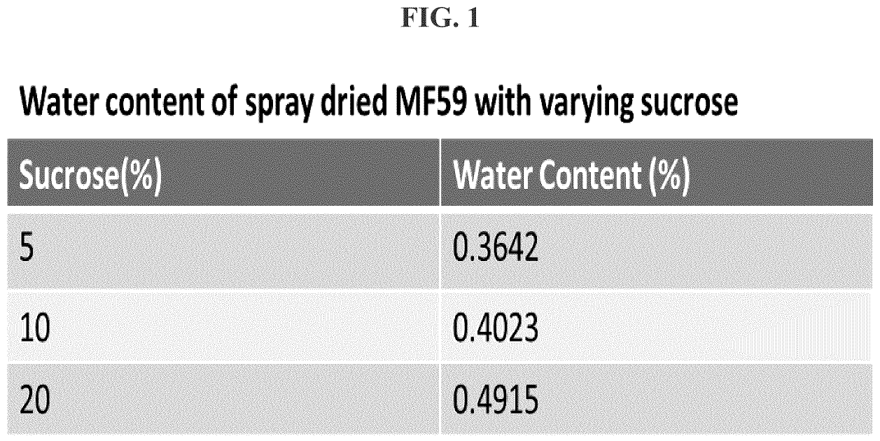

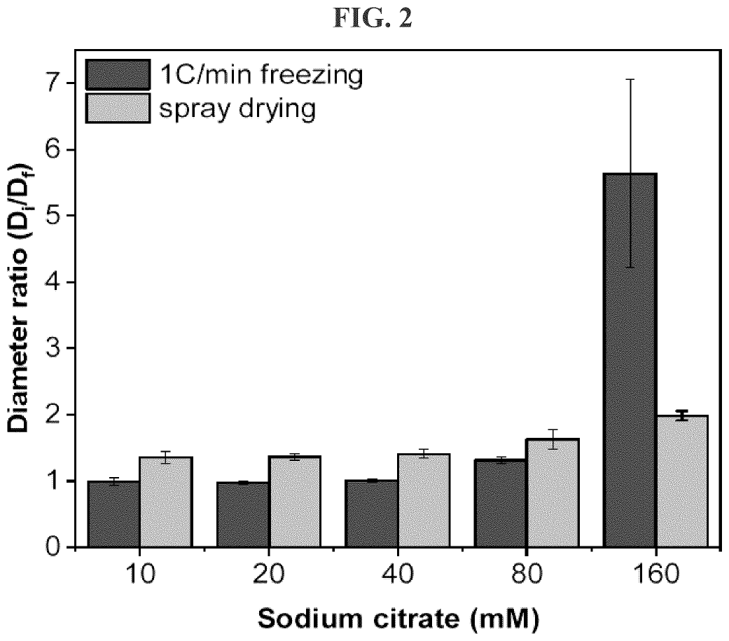

As noted herein, emulsion samples with about 20% w/v polysaccharide (e.g., sucrose) were also frozen with varying starting concentrations of a representative salt (e.g., sodium citrate salt). In this example, at a sodium citrate concentration of 80.0 mM, there was a slight increase in the emulsion droplet diameter after freezing, with a larger increase in droplet diameter in the sample with 160.0 mM sodium citrate (FIG. 2). In order to decouple effects of the concentration of solutes during freezing from causing damage due at least in part to ice crystallization, an alternative drying technique, spray-drying, was implemented. During spray-drying, solutes become more and more concentrated as the atomized spray-dried particles dry, avoiding a freezing step that typically introduces ice crystallization and side effects. Powders formed by spray-drying had residual water contents of less than 1% (FIG. 1. Table). Higher concentrations of salt destabilized the emulsions during spray-drying, where starting salt concentrations of 80.0 and 160.0 mM led to increases in emulsion droplet diameters as instabilities caused droplet coalescence. Spray dried emulsion samples at 10.0, 20.0-and 40.0-mM sodium citrate all retained an emulsion droplet diameter after reconstitution of about 250 nm, while the samples with 80-and 160.0-mM sodium citrate had emulsion droplet diameters of 300 and 360 nm, respectively. Surprisingly, the destabilizing effect of drying-induced salt increase in concentration was reduced or avoided when spray-drying was used to effect drying avoiding a freezing step, in contrast to the dramatic destabilization observed during lyophilization-based drying having a freezing step.

FIG. 1, Table 1. Represents water content of spray-dried powders formed by spray-drying a representative squalene-based lipid adjuvant nanoemulsion to form solutions containing varying amounts of sucrose.

FIG. 2 is a plot demonstrating effect of sodium citrate concentration on droplet diameters of emulsion samples that were frozen (dark bars) or spray dried (light bars). Emulsion concentrations were at 1.25% v/v squalene and sucrose concentrations were at 20% w/v in the presence of increasing salt concentrations (e.g., sodium citrate). Error bars shown are the standard deviation from the average of 3 replicates. Di=diameter before freezing or spray drying, Dr=diameter after freezing or spray drying.

### Example 2

Emulsion Destabilization During Powder Formation by drying of Squalene Adjuvant Emulsions

In another exemplary method, another issue during freezing was analyzed to assess the effect of spray-drying under various conditions on lipid emulsion compositions. This other stress occurs during freezing in addition to damage due to crystallization is solute concentration. One of the solutes in the emulsion formulations is a salt (e.g., sodium citrate salt). Increases in salt such as sodium citrate concentration during freezing has the potential to shield charges between emulsion droplets and lead to lower zeta potential values that would in turn facilitate particle-particle interactions between droplets leading to adverse effects on agent-containing lipid emulsions.

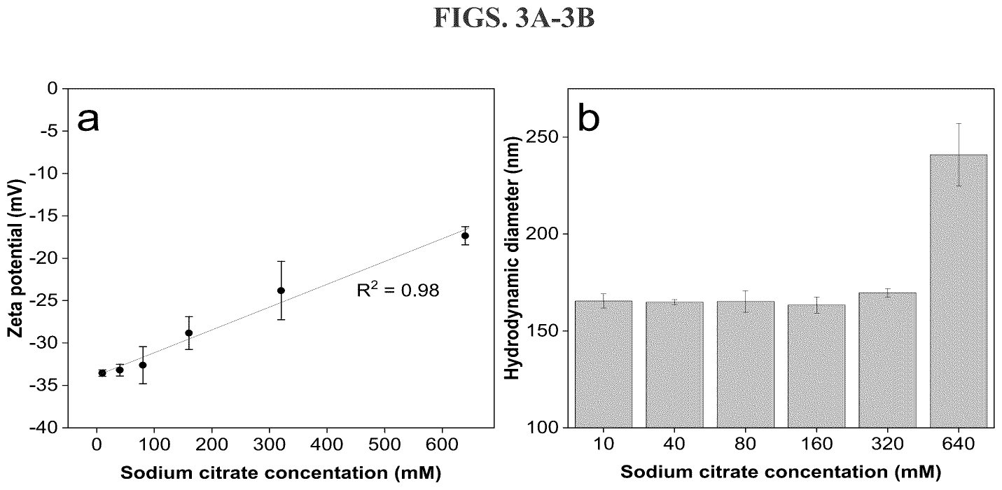

To determine how increased salt concentrations affect zeta potential values, emulsion samples were prepared with various concentrations of sodium citrate, a salt, at pH 7.0. Because polysaccharides (e.g., sucrose) maximal concentration is about 80% w/v at its glass transition temperature, the starting polysaccharide concentration (e.g., sucrose) dictates the extent of concentration that the rest of the solutes undergo. Therefore, the concentrations of salt investigated represent the maximum concentration of salt (e.g., sodium citrate) that would be present in the freeze-concentrate for the starting polysaccharide (e.g., sucrose) concentrations of 1.25, 2.5, 5, 10, and 20% w/v. Zeta potential values decreased in magnitude correlating with increasing concentrations of salt (e.g., sodium citrate) (FIG. 3A), indicating that charge screening in concentrated salt solutions reduces electrostatic repulsion between emulsified lipid-based adjuvant nanodroplets (e.g., squalene). For samples with salt (e.g., sodium citrate) concentrations up to and including 320 mM but less than 640 mM (FIG. 3B), there was no dependence on zeta potential and diameter, which were all around 170 nm. At a starting sucrose concentration of 1.25% w/v, the extent of concentration for the system to reach the 80% w/v disaccharide (e.g., sucrose) threshold at the glass transition temperature would be about 64x; therefore, a maximum salt concentration of about 640 mM salt (e.g., sodium citrate) in the freeze-concentrated liquid. For the sample formulated at the maximum tested concentration of 640 mM salt (e.g., sodium citrate), creaming or turbidity of the emulsion was visible almost immediately after formulation and there was an increase in hydrodynamic diameter to 240 nm (FIG. 3B).

FIG. 3A-3B illustrate effect of salt (e.g., sodium citrate) concentration in aqueous liquid emulsions of an exemplary lipid emulsion of squalene-based adjuvant for 3A) zeta potential and 3B) emulsion droplet diameter. Emulsion concentrations were at 1.25% v/v squalene. Error bars shown are the standard deviation from the average of 3 replicates.

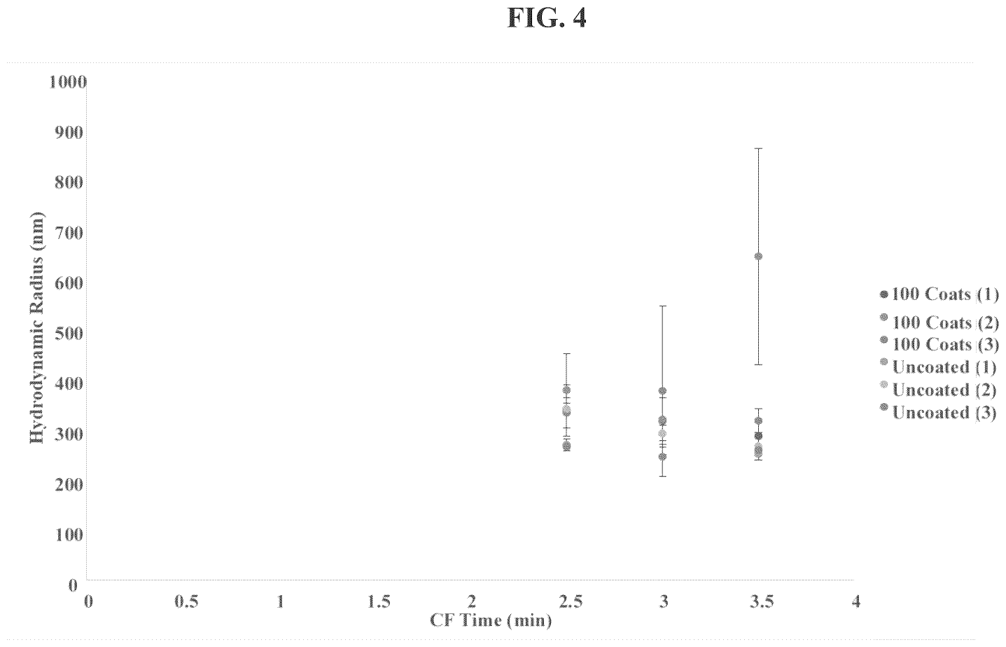

FIG. 4. Illustrates an exemplary lipid-emulsion (Squalene-based adjuvant emulsion) droplet size distributions in uncoated, reconstituted spray-dried powders compared to an exemplary lipid-emulsion (Squalene-based adjuvant emulsion) droplet size distributions measured in samples that had been spray-dried and coated with about 100 layers of trimethyl alumina using ALD, and suspended in buffer (e.g., EDTA/His) to remove coating and resuspend the emulsion droplets. All samples were subjected to brief centrifugation, which caused any undissolved alumina coating fragments to pellet. Samples taken from the supernatant following centrifugation were analyzed by dynamic light scattering to determine the average droplet size. Droplet size distributions for emulsions in powders with 100 layers of alumina applied by atomic layer deposition were indistinguishable from those in powders without coatings demonstrating efficacy of these methods without altering microparticle droplet sizes.

### Example 3

Stability of emulsified squalene-based adjuvants in spray-dried powders coated with atomic layer-deposited alumina

In another exemplary method, lipid emulsions (e.g., squalene-based lipid droplet emulsions) were spray-dried from solutions containing a representative disaccharide and salt in an exemplary concentration (e.g., 20% sucrose, 20 mM sodium citrate). Analysis of the powders formed by spray-drying demonstrated a water content of less than about 1.0%. The sample was divided into two aliquots. One aliquot of powder was retained and poured into glass vials which were evacuated to 60 mTorr, backfilled to atmospheric pressure with dry nitrogen, and sealed. For the “coated” samples, powders were placed in a custom-built ALD fluidized bed reactor, where 100 cycles of ALD with alternating injection of water vapor and trimethyl aluminum gas were used to apply 100 molecular layers of alumina onto the surface to the remaining aliquoted samples.

To test whether the droplet size within the exemplary MF-59-like emulsion was altered during ALD application of alumina to the surface of the powders, the alumina-coated powders were suspended in 30 mM EDTA, 5 mM His, pH 6, dissolving a sufficient amount of the alumina coating to allow the internal contents of the coated powders to be released into the solution.

Brief centrifugation was applied to the suspension, causing any remaining alumina shells that had been coating the powders to sediment. Samples of the supernatant that contained the released interior contents of the powders were examined by dynamic light scattering to determine the size distribution of the released exemplary MF59®-like emulsions. Size distributions of emulsified droplets were compared with size distributions of droplets from reconstituted powders that had not been coated by atomic layer deposition. As illustrated in FIG. 4, the droplet size distributions before and after ALD of 100 molecular layers of alumina were unchanged.

### Example 4

Polysaccharide concentration for stabilizing squalene-based lipid emulsion

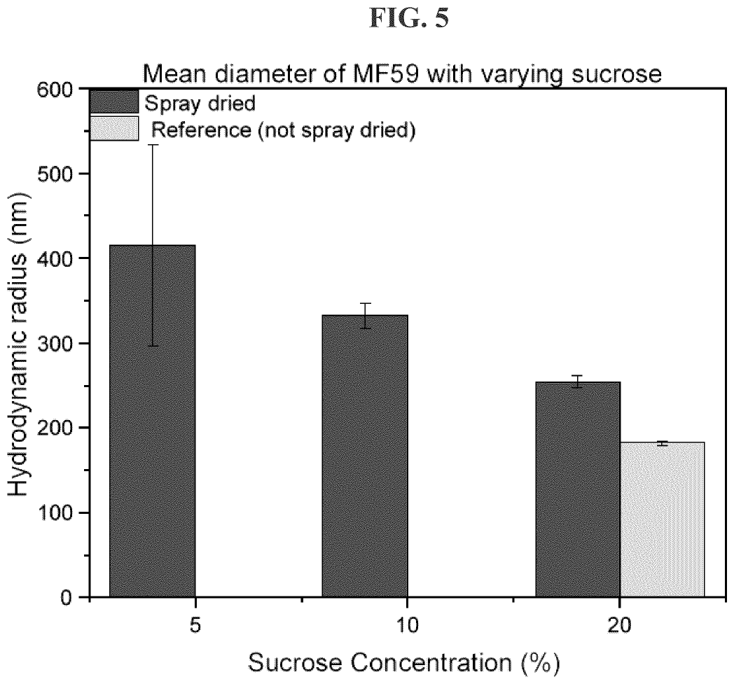

FIG. 5 illustrates size distribution of emulsified squalene after reconstitution of spray dried powder. Maintenance of droplet size below 300 nm requires polysaccharide (e.g., sucrose) concentration in sprayed liquid to be at least about 10%, or at least about 20% or more but less than 40% w/v.

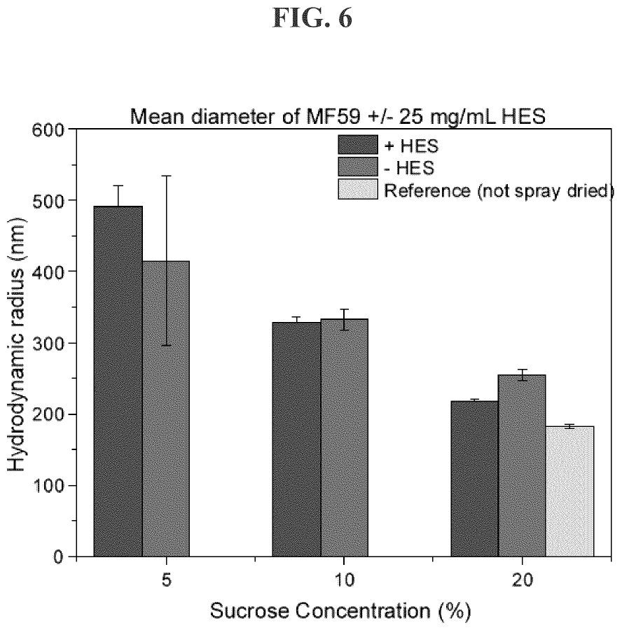

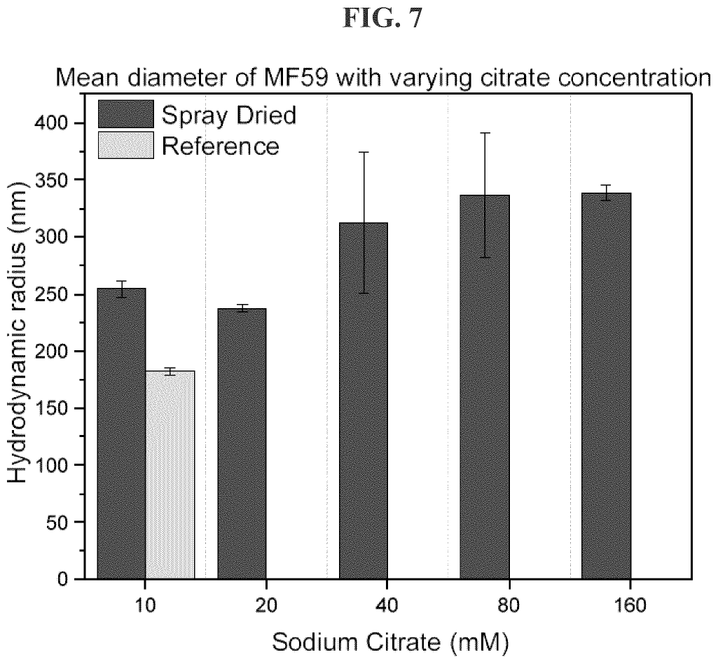

FIG. 6 illustrates optimal retention of initial lipid emulsion size distribution after spray-drying and reconstitution which is demonstrated to be enhanced by the addition of an additional glass-forming agent (e.g., hydroxyethyl starch) to the solution to be spray-dried. FIG. 7. illustrates optimal retention of initial lipid emulsion size distribution after spray-drying and reconstitution requires ionic strength below about 100 mmole/L (e.g., 20 mM sodium citrate) to avoid coalescence during spray drying.

All the compositions and methods disclosed and claimed herein can be made and executed without undue experimentation in light of the present disclosure. While the compositions and methods have been described in terms of embodiments, it is apparent to those of skill in the art that variations maybe applied to the compositions and methods and in the steps or in the sequence of steps of the methods described herein without departing from the concept, spirit
and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. An atomic layer deposition (ALD) coated antigen or agent-containing microparticle comprising:
a central or innermost antigen or agent-containing lipid-emulsion containing glassy microparticle comprising at least one agent, at least one antigen or combination thereof in a lipid emulsion and at least one glass-forming agent; and
one or more coating layers comprising one or more of a metallo-organic material, metal oxides, metal alkoxides, and/or aluminum-based coating layer covering the central or innermost antigen or agent-containing lipid-emulsion containing glassy microparticle.

2. The microparticle according to claim 1, wherein the glass-forming agent comprises at least one polysaccharide.

3. The microparticle according to claim 2, wherein the at least one polysaccharide to lipid concentration of the lipid-emulsion comprises a ratio sufficient to stabilize the lipid-emulsion during spray-drying; optionally, wherein the ratio is greater than 5:1; 6:1, 7:1, or at least 8:1 polysaccharide to lipid.

4. The microparticle according to claim 1, further comprising at least one additional glass-forming agent or polysaccharide; optionally, wherein the at least one additional glass-forming agent or polysaccharide comprises at least one of hydroxyethyl starch, dextran, carboxymethyl cellulose, or viastarch.

5. (canceled)

6. The microparticle according to claim 1, wherein the at least one agent or at least one antigen comprises an agent or antigen derived from a pathogenic organism.

7. The microparticle according to claim 6, wherein the at least one agent or the at least one antigen comprises a polynucleotide, a polypeptide, a recombinant protein; a virus-like particle; a live virus; a live, attenuated virus; an inactivated virus; a toxoid; or a combination thereof.

8. The microparticle according to claim 1, wherein the at least one agent or the at least one antigen comprises one or more antigens derived from a pathogenic virus, pathogenic bacteria, pathogenic prion, pathogenic fungi, pathogenic protozoa, worm, pathogenic yeast, or other pathogen.

9. The microparticle according to claim 1, wherein the at least one agent or the at least one antigen comprises at least one antigen or at least one agent derived from a category A, category B or category C pathogen from the National Institute of Allergy and Infectious Diseases (NIAID) listings.

10. The microparticle according to claim 1, wherein the at least one agent or the at least one antigen comprises at least one antigen derived from at least one of Bacillus anthracis (anthrax), Clostridium botulinum toxin (botulism), Yersinia pestis (plague), Variola major (smallpox) and other related pox viruses, Francisella tularensis (tularemia), viral hemorrhagic fevers, Arenaviruses, Junin, Machupo, Guanarito, Chapare, Lassa, Lujo, bunyaviruses, hantaviruses causing Hanta pulmonary syndrome, Rift Valley Fever, Crimean Congo Hemorrhagic Fever, flaviviruses, alphaviruses, and Filoviruses, or a combination thereof.

11. The microparticle according to claim 8, wherein the at least one antigen is derived from at least one pathogen, wherein the at least one pathogen comprises at least one of human papilloma virus (HPV), Ebola virus, Marburg virus, poliovirus, norovirus, rotavirus, hepatitis A virus, hepatitis, B virus, hepatitis C virus, varicella-zoster virus, herpes simplex virus, cytomegalovirus, dengue virus, Yellow fever virus, Japanese encephalitis virus, West Nile virus, Zika virus, Haemophilus influenzae type b virus, measles virus, mumps virus, rubella virus, respiratory syncytial virus, influenza virus, rabies virus, smallpox virus, parvovirus, chikungunya virus, Corynebacterium diptheriae, Clostridium tetani, Clostridium botulinum, Bordetella pertussis, Streptococcus pneumoniae, Neisseria meningitides, Salmonella spp., Bacillus anthracis, Yersinia spp., or a combination thereof.

12. The microparticle according to claim 8, wherein the at least one antigen is derived from at least one pathogen, wherein the at least one pathogen comprises a pathogen that infects a companion animal, livestock, birds, or other non-human animal and the pathogen comprises at least one of canine parvovirus, canine distemper virus, canine adenovirus, rabies virus, canine parainfluenza virus, canine influenza virus, canine corona virus, West Nile virus, Eastern Equine Encephalitis virus (EEEV), Western Equine Encephalitis virus (WEEV), Venezuelan Equine Encephalitis virus (VEEV), chikungunya virus, equine influenza virus, equine herpesvirus, Streptococcus equi equi, Clostridium tetani, Neorickettsia risticii, Clostridium tetani, measles virus, Bordetella bronchiseptica, Leptospira spp., Borrelia burgdorferi, feline herpesvirus 1, feline calicivirus, feline panleukopenia virus, rabies virus, feline leukemia virus, feline immunodeficiency virus, virulent systemic feline calicivirus, Chlamydophila felis, Pasteurella haemolytica, Bordetella bronchiseptica, bovine herpesvirus, parainfluenza type 3 virus, bovine viral diarrhea virus, bovine respiratory syncytial virus, Clostridium chauvoei, Clostridium septicum, Clostridium novyi, Clostridium perfringens type C, Clostridium perfringens type D, Pasteurella haemolytica, Clostridium haemolyticum, Chikungunya virus, Marek's disease virus, reovirus, avian encephalomyelitis virus, avian influenza virus, avipoxviruses, chicken anemia virus, Pasteurella multocida, Newcastle disease virus, Riemerella anatipestifer, duck herpesvirus 1, duck hepatitis virus, or a combination thereof.

13-14. (canceled)

15. The microparticle according to claim 8, wherein the at least one antigen is derived from at least one pathogen, wherein the at least one pathogen comprises at least one of Cryptococcus spp., Aspergillus spp., Blastomyces spp., Candida albicans, Paracoccidioides spp., Sporothrix spp., Histoplasma capsulatum, Pneumocystis jirovecii, Coccidioides immitis, or a combination thereof.

16. The microparticle according to claim 1, wherein the at least one agent comprises a multimeric protein complex.

17. The microparticles according to claim 1, wherein the antigen or agent is derived from a virus and the virus comprises an enveloped or non-enveloped virus.

18. The microparticles according to claim 1, wherein the antigen or agent is derived from a virus and the virus comprises a positive or negative strand RNA virus, a linear or circular double-stranded or single-stranded DNA virus, or an RNA/DNA hybrid virus or an mRNA encoding a virus or viral antigen or an mRNA encoding a therapeutic protein, a cell receptor protein, an enzyme, a structural protein, a cytokine or a combination thereof.

19. The microparticle according to claim 1, wherein the at least one glass-forming agent comprises at least one of trehalose, sucrose, ficoll, dextran, maltotriose, lactose, mannitol and glycine, hydroxyethyl starch, polyvinylpyrrolidone, glycine, cyclodextrin, povidone and a combination thereof.

20. (canceled)

21. The microparticle according to claim 1, further comprising at least one salt.

22. The microparticle according to claim 1, wherein each layer of the one or more coating layers comprises one or more of aluminum oxide (Al₂O₃), an aluminum alkoxide, silicon dioxide (SiO₂), Zinc dioxide (ZnO₂), titanium dioxide (TiO₂), and silicon nitride (Si₃N₄) or combinations thereof.

23-25. (canceled)

26. A method of making an antigen-or agent-containing lipid-emulsion microparticle according to claim 1, the method comprising:
a) combining at least one agent or at least one antigen or combination thereof in a lipid emulsion and at least one glass-forming agent; and
b) spray-drying a) to make essentially dried agent-containing or antigen-containing lipid emulsion-containing microparticles to generate a central or innermost antigen-or agent-containing lipid-emulsion; and
c) coating by ALD, the essentially dried agent-containing or antigen-containing lipid emulsion-containing microparticles of b) with one or more coating layers comprising one or more of a metallo-organic material, metal oxides, metal alkoxides, and/or aluminum-based coating layer encasing the central or innermost essentially dried agent-containing or antigen-containing lipid emulsion-containing microparticles.

27. The method according to claim 26, wherein the at least one agent or at least one antigen comprises at least one agent or at least one antigen derived from a pathogen.

28. The method according to claim 26, wherein the at least one agent or at least one antigen comprises a recombinant peptide, a polynucleotide, a polypeptide, a recombinant protein, a virus-like particle, a live virus, a live, attenuated virus, an inactivated virus, a toxoid, a pathogenic virus, a pathogenic bacterium, a pathogenic prion, a pathogenic fungus, a pathogenic protozoan, or a combination thereof.

29. (canceled)

30. The method according to claim 26, wherein the ratio of concentration of polysaccharide to lipid-emulsion agent is at least 5:1, or at least 6:1, or at least 7:1, or at least 8:1, or at least 9:1 or at least 10:1.

31. A microparticle-containing composition according to claim 1, wherein the microparticle-containing composition comprises a plurality of microparticles.

32. The microparticle-containing composition according to claim 31, further comprising at least one pharmaceutically acceptable excipient.

33. (canceled)

34. A method for treating, or reducing the risk of onset of a health condition in a subject, the method comprising administering to the subject a composition according to claim 32 to the subject.

35. (canceled)

36. A kit comprising at least one microparticle according to claim 1, and at least one container.

37. The microparticle according to claim according to claim 1, wherein the lipid emulsion comprises a triterpenoid.

38. The microparticle according to claim 1, wherein the lipid emulsion comprises squalene oil.
